(19) United States
(12) Patent Application Publication
(10) Pub. No.: US 2003/0171276 A1
(43) Pub. Date: Sep. 11, 2003

(54) Preventives and remedies for chronic hepatitis

(76) Inventors: Naoki Tohdoh, Hyogo (JP); Masashi Murata, Osaka (JP); Takashi Enjoji, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/169,297

(22) PCT Filed: Dec. 28, 2000
(86) PCT No.: PCT/JP00/09393

(30) Foreign Application Priority Data
Dec. 28, 1999 (JP) 11/374087

(51) Int. Cl. A61K 38/17

(52) U.S. Cl. 514/12

(57) ABSTRACT

To provide a prophylactic or therapeutic agent for chronic hepatitis, capable of inhibiting the binding of hepatitis virus and a target cell, thereby preventing or treating chronic hepatitis; and a fusion interferon capable of still more improving a viral clearance ratio at the termination of interferon administration. A prophylactic or therapeutic agent for chronic hepatitis, comprising an oligopeptide having the following characteristics: (A) having a binding affinity to a viral antigen protein; (B) inhibiting binding of virus to a receptor protein on a target cell for said virus; and (C) having homologies with said receptor protein at the amino acid sequence level; a fusion interferon containing an amino acid sequence of the above-mentioned oligopeptide; and method for treating chronic hepatitis in combination with the use of interferon, characterized by using the oligopeptide or a part thereof in combination with interferon.

### TECHNICAL FIELD

The present invention relates to a therapeutic agent for chronic hepatitis, comprising an oligonucleotide having an amino acid sequence or a part thereof, wherein the amino acid sequence is important for infection of hepatitis virus into a target cell. Concretely, the present invention relates to a therapeutic agent for chronic hepatitis, comprising an oligopeptide or a part thereof, the oligopeptide being capable of binding to an antigenic protein for hepatitis virus and having an action of inhibiting binding of virus to a receptor protein, or a compound prepared on the basis of its sequence. Also, the present invention relates to a fusion interferon comprising the oligopeptide and its partial amino acid sequence. Further, the present invention relates to a method for treating chronic hepatitis in combination with the use of interferon.

### BACKGROUND ART

Chronic type-B hepatitis and type-C hepatitis developed by infection with hepatitis B virus (HBV) and hepatitis C virus (HCV) are grave viral diseases which are transformed into cirrhosis or hepatocellular carcinoma at a high ratio. In Japan, the number of hepatitis B virus carriers is said to be about one million, and that of hepatic C virus carriers is said to be about 1.3 millions. The carriers have been spread world-widely. HCV, which is a single-stranded RNA virus belonging to Flaviviridae, has been considered to transform to chronicity by factors for the virus side such as avoidance from the immune mechanism by gene mutation and factors from the host side such as decrease in the immunocompetence and the viral clearance ability. On the other hand, since HBV belonging to Hepadonaviridae is a double-stranded DNA virus, a mutation in a virus genome does not take place as much as it does in HCV; however, it transforms to chronicity by incorporation into chromosome DNA. Also in HBV, there is found fulmination caused by avoidance from the immune mechanism caused by mutation of a virus gene.

Presently, the only one treatment method for eliminating the virus is an interferon treatment method. However, a ratio at which the virus is finally eliminated is 40% or so in HCV and 20% or so in HBV. In particular, in an interferon treatment method for the HCV, there is caused a difference in its therapeutic effect depending upon the status of the patient, such as serotype of HCV, virus amount and histological progression degree. In particular, in the case of genotype II and hyperviremia, it has been known that its amelioration effect is low and a virus clearance ratio is also low.

As a method of treating chronic type-C hepatitis, a treatment mainly comprising clearance of hepatitis C virus (HCV) and improvement in liver function is carried out. Inter alia, a treatment of chronic type-C hepatitis by interferon having improved actions in both HCV clearance and liver function is carried out as the only one method of treatment for virus clearance. However, such a treatment has the disadvantage that a virus complete clearance ratio for HCV is as low as 40% or so. For that reason, a method for treating chronic hepatitis in combination with the use of interferon and ribavirin, amantadine or the like has been recently developed; however, the method still has a disadvantage that the effectiveness is yet low. Alternatively, a drug for improving a liver function by using glycyrrhizin or the like has been tried for a patient showing resistance to interferon; however, the drug has a disadvantage that the liver function is rapidly worsened after the termination of administration.

On the other hand, as a method of treating chronic type-B hepatitis, an interferon treatment method is carried out as in the case of chronic type-C hepatitis; however, the therapeutic effect can be expected currently on the level of only 20% or so. Recently, in the treatment of chronic type-B hepatitis, an inhibitor for an HBV polymerase has been administered; however, the administration has the large side effects and relapse of virus is found after the termination of administration. Further, upon the development of fulminant type-B hepatitis, a method for treatment in combination with the use of interferon and cyclosporin is carried out after exchange of whole serum; however, a lifesaving rate is low currently. As described above, a treatment method which is truly effective for chronic type-B hepatitis and chronic type-C hepatitis which may lead to development of cirrhosis or hepatocellular carcinoma has not been established currently, even though hepatitis B virus and hepatitis C virus have been distributed world-widely and virus carriers are present at 1% or so or greater of the population.

Accordingly, the present invention has been accomplished in view of the above-mentioned conventional problems, and a first object of the present invention is to provide a prophylactic or therapeutic agent for chronic hepatitis, capable of inhibiting the binding of hepatitis virus and a target cell, thereby preventing or treating chronic hepatitis. A second object of the present invention is to provide a fusion interferon and a method for treating chronic hepatitis in combination with the use of interferon, capable of still more improving a viral clearance ratio at the termination of interferon administration.

### DISCLOSURE OF INVENTION

Concretely, the gist of the present invention relates to:

[1] a prophylactic or therapeutic agent for chronic hepatitis, comprising an oligopeptide having the following characteristics:

(A) having a binding affinity to a viral antigen protein;

(B) inhibiting binding of virus to a receptor protein on a target cell for the virus; and

(C) having homologies with the receptor protein at the amino acid sequence level;

[2] a prophylactic or therapeutic agent for chronic type-C hepatitis, comprising a compound having the following characteristics:

(a) having a binding affinity to an envelope protein of HCV; and

(b) inhibiting binding of HCV to a receptor protein on a target cell for the HCV;

[3] a prophylactic or therapeutic agent for chronic type-B hepatitis, comprising a compound having the following characteristics:

(a′) having a binding affinity to S antigen protein of HBV; and

(b′) inhibiting binding of HBV to a receptor protein on a target cell for the HBV;

[4] a fusion interferon comprising an amino acid sequence of an oligopeptide having the following characteristics:

(A) having a binding affinity to a viral antigen protein;

(B) inhibiting binding of virus to a receptor protein on a target cell for the virus; and

(C) having homologies with the receptor protein at the amino acid sequence level; and

[5] a method for treating chronic hepatitis in combination with the use of interferon, characterized by using an oligopeptide or a part thereof in combination with interferon, wherein the oligopeptide has the following characteristics:

(A) having a binding affinity to a viral antigen protein;

(B) inhibiting binding of virus to a receptor protein on a target cell for the virus; and

(C) having homologies with the receptor protein at the amino acid sequence level.

### BRIEF DESCRIPTION OF THE DRAWINGS

FIG. 1 shows a schematic view of a construct of a gene for interferon with a corresponding oligopeptide added on a C-terminal side.

FIG. 2 is a diagram showing the analytical results on the homologies between an amino acid sequence of human CD81 and that of bovine lactoferrin. In Panel A, an upper row shows an amino acid sequence of human-derived CD81, and a lower row an amino acid sequence of bovine lactoferrin, and a homologous region between human CD81 and bovine lactoferrin is shaded with meshed pattern. Panel B shows a region which is mostly conserved in the homologous region shown by Panel A.

FIG. 3 is a diagram showing the analytical results on the homologies between an amino acid sequence of bovine lactoferrin and that of human lactoferrin. An upper row shows bovine lactoferrin and a lower row shows human lactoferrin. In the figure, a region showing the homologies with CD81 is shown.

FIG. 4 shows a diagram predicting secondary structure of a partial amino acid sequence of a homologous sequence between human CD81 and bovine lactoferrin, which are considered to be bound to HCV. In the figure, among the homologous sequences, the identical amino acids are indicated by bold characters, and the homologous amino acids are indicated by plain characters. In addition, in the prediction of the secondary structure, a part corresponding to amino acids constituting the same secondary structure between human CD81 and bovine lactoferrin is shaded with meshed pattern. In the case of human lactoferrin, a sequence homologous to human CD81 and the identical portions of amino acids maintaining the common secondary structure between human CD81 and human lactoferrin in the amino acid sequence are shaded with meshed pattern. Panel A shows a binding site of human CD81, Panel B shows a binding site of bovine lactoferrin, and Panel C shows a binding site of human lactoferrin.

FIG. 5 is a diagram showing the analytical results on the homologies of an amino acid sequence of a binding region, on the basis of an amino acid sequence of duck-derived carboxypeptidase D and duck-derived glycine decarboxylase complex P protein, in which binding to duck HBV pre-S antigen is suggested. Panel A shows the analytical results on N-terminal side, and Panel B shows the analytical results on C-terminal side.

FIG. 6 shows a diagram predicting a secondary structure of the homologous region in FIG. 5. Panel A shows the analytical results on N-terminal side, and Panel B shows the analytical results on C-terminal side.

FIG. 7 shows a diagram showing the analytical results on the homologies between an amino acid sequence of a binding region of duck-derived carboxypeptidase D to duck HBV and an amino acid sequence of human-derived carboxypeptidase D.

FIG. 8 shows a diagram showing the analytical results on the homologies between an amino acid sequence of a binding region of duck-derived carboxypeptidase D to duck HBV and amino acid sequence of human-derived carboxypeptidase D. FIG. 8 is a continuation from the analytical results shown in FIG. 7.

FIG. 9 shows the analytical results on the homologies between an amino acid sequence of duck-derived glycine decarboxylase complex P protein and an amino acid sequence of human-derived glycine decarboxylase complex P protein.

FIG. 10 is a diagram showing the analytical results on the homologous regions of human CD81, bovine lactoferrin, human lactoferrin and human LDL receptor. C circumscribed by an open square means a cysteine residue which is commonly found in all the sequences. Basic amino acids contained in respective sequences are shown by bold characters. Amino acid residues which are homologous in three of the sequence of human CD81 (SEQ ID NO: 1), the sequence of bovine lactoferrin (SEQ ID NO: 2) and the sequence of human lactoferrin (SEQ ID NO: 3) are shaded with meshed pattern. In the sequence of human LDL receptor (SEQ ID NO: 18) in an upper row, amino acid residues which are homologous to those of human CD81 are shaded with meshed pattern. In the sequence of human LDL receptor (SEQ ID NO: 19) in a lower row, residues which are homologous to those of human lactoferrin are shaded with meshed pattern.

FIG. 11 is a diagram showing the analytical results on a homologous region between an amino acid sequence of human CD81 and that of human LDL receptor.

FIG. 12 is a diagram showing the analytical results on a homologous region between an amino acid sequence of human lactoferrin and that of human LDL receptor.

FIG. 13 is diagrams showing the results of CD81 expression analyzed by FACScan. Panel (1) is a histogram obtained by staining Jurkat cells with an anti-human CD81 antibody and an FITC-labeled anti-mouse IgG1 antibody, and analyzing by FACScan. A horizontal axis represents FITC fluorescence intensity and a vertical axis represents the number of cells. The results of staining with an isotype control antibody (mouse IgG1), which is a negative control in staining, is shown by a fine solid line, and the results of staining with an anti-human CD81 antibody is shown by a bold solid line. On the other hand, Panel (2) is a histogram obtained by analyzing binding of the recombinant E2 protein to the Jurkat cells by FACScan. The results of staining with the mouse IgG1 is shown by a fine solid line; the results obtained by staining the Jurkat cells untreated with the recombinant E2 protein, with an anti-E2 antibody is shown by a dotted line; and the results obtained by staining the Jurkat cells treated with the recombinant E2 protein, with an anti-E2 antibody is shown by a bold solid line.

FIG. 14 is a histogram obtained by analyzing with FACScan the binding to the Jurkat cells of the recombinant E2 protein previously incubated with the human CD81-derived peptide [Panel (1)], the bovine lactoferrin protein [Panel (2)] or the bovine lactoferrin-derived peptide [Panel (3)]. The results for which the Jurkat cells without treatment of the recombinant E2 protein were stained with an anti-E2 antibody is shown by a thin solid line, the results for which the Jurkat cells treated with the recombinant E2 protein alone were stained with the anti-E2 antibody is shown by a bold solid line, and the results for which the Jurkat cells treated with the recombinant E2 protein preincubated with a peptide or a protein were stained with the anti-E2 antibody is shown by a dotted line.

FIG. 15 is a diagram showing the procedures for constructing respective expression vectors for IFN-α2b, IFN-CD81, IFN-human lactoferrin and IFN-bovine lactoferrin, which are HCV binding type IFNs.

FIG. 16 is a schematic diagram showing the results of Western blot analysis with an anti-IFN-α antibody, regarding IFN-α2b, and IFN-CD81, IFN-human lactoferrin and IFN-bovine lactoferrin, which are HCV binding type IFNs, wherein each of them is obtained by protein synthesis with in vitro transcription/translation by using the prepared expression vector as a template. Lane 1: pCIA, lane 2: pCIA-CD81, lane 3: pCIA-HLFN, lane 4: pCIA-BLFN, and lane 5: Luciferase T7 control DNA. A solid black arrow shows IFN-α2b, and an open arrow shows the HCV binding type IFNs.

FIG. 17 is a schematic diagram showing the results of Western blot analysis with an anti-IFN-α antibody, regarding IFN-α2b, and IFN-CD81, IFN-human lactoferrin and IFN-bovine lactoferrin, which are HCV binding type IFNs, wherein each of them is obtained by transient expression in COS-1 cells. Lane 1: pCIA, lane 2: pCIA-CD81, lane 3: pCIA-HLFN, and lane 4: pCIA-BLFN. A solid black arrow shows IFN-α2b, and an open arrow shows the HCV binding type IFNs.

FIG. 18 is a diagram showing the anti-viral activity of the culture supernatant of COS-1 cells three days after transfection of pCIA and pCIA-BLFN. In the system for evaluating the Sindbis virus-FL cells, the evaluation was carried out by a Crystal Violet method. INTRON A was 2-fold stepwise diluted where 200 IU/ml was regarded as a dilution fold 1. The culture supernatants were 2-fold stepwise diluted where 1000-fold dilution with the medium was regarded as a dilution fold 1. A vertical axis shows the absorbance of the cells stained with Crystal Violet at 595 nm.

FIG. 19 is a diagram showing the anti-viral activity of the culture supernatant of the COS-1 cells three days after transfecion of pCIA-CD81 and pCIA-HLFN. In the system for evaluating the Sindbis virus-FL cells, the evaluation was carried out using a Crystal Violet method. INTRON A was 2-fold stepwise diluted where 200 IU/ml was regarded as a dilution fold 1. The culture supernatants were 2-fold stepwise diluted where 1000-fold dilution with the medium was regarded as a dilution fold 1. A vertical axis shows the absorbance of the cells stained with Crystal Violet at 595 nm.

FIG. 20 is a schematic diagram showing the binding of transiently expressed HCV binding type IFNs in COS-1 cells to baculovirus-derived recombinant GST-fusion HCV E2 protein (GST-E2). Each of GST-E2 and IFN-α2b (lanes 1 and 5), IFN-CD81 (lanes 2 and 6), IFN-human lactoferrin (lanes 3 and 7), and IFN-bovine lactoferrin (lanes 4 and 8), which are HCV-binding type IFNs, was incubated, and co-precipitated with Glutathione beads, and thereafter the detection was carried out using an anti-IFN-α antibody. Lanes 1 to 4 are precipitation fractions, and lanes 5 to 8 are supernatant fractions. A solid black arrow shows IFN-α2b, and an open arrow shows HCV-binding type IFNs.

### MODES FOR CARRYING OUT THE INVENTION

One of the features of the prophylactic or therapeutic agent for chronic hepatitis of the present invention resides in that the prophylactic or therapeutic agent comprises an oligopeptide having the following characteristics:

(A) having a binding affinity for a viral antigen protein;

(B) inhibiting binding of virus to a receptor protein on a target cell for the virus; and

(C) having homologies with the receptor protein at the amino acid sequence level.

Since the prophylactic or therapeutic agent for chronic hepatitis of the present invention comprises the oligopeptide having the above-mentioned characteristics (A) to (C), it is considered that the binding of hepatitis virus, especially hepatitis C virus or hepatitis B virus, to a target cell is inhibited. Therefore, there are exhibited excellent effects that chronic hepatitis can be prevented or treated.

In the present specification, hepatitis C virus will be abbreviated as “HCV” and hepatitis B virus will be abbreviated as “HBV.”

The viral antigen protein includes, for instance, an envelope protein of HCV, pre-S protein of HBV, and the like. In addition, the concept of the present invention can be applied to diseases other than chronic hepatitis. In this case, the envelope protein of HIV or the like can be used as a viral antigen protein.

Since the oligopeptide having the above-mentioned characteristics (A) to (C) comprises an amino acid sequence essential for binding of a receptor protein to virus, the binding affinity of a viral antigen protein to an oligopeptide is exhibited. It is desired that the above-mentioned binding affinity is higher than the binding affinity of virus to a receptor protein on a target cell for the virus, from the viewpoint of inhibiting the binding of the virus to the receptor protein on the target cell for the virus.

The receptor protein of a target cell for the virus includes human CD81, which is a receptor molecule for HCV; carboxypeptidase D, to which pre-S protein of HBV is deduced to be bound; glycine decarboxylase complex P protein and the like. In the present invention, human CD81, human carboxypeptidase D and glycine decarboxylase complex P protein are preferable.

At the amino acid sequence level, the above-mentioned oligopeptide has the homologies with a receptor protein of 20% or more at a homologous amino acid level, from the viewpoint of exhibiting sufficient binding affinity. In addition, in the case where similar amino acids are included, it is preferable that the homologies are 50% or more.

The present invention has been accomplished on the basis of the technical idea described below.

With respect to HCV, which is an etiologist for hepatitis C, it has been reported that some antibodies to a hypervariable region (HVR1) which is a part of E2 region of an envelope protein of HCV weakens the infectivity of virus (Rosa D. et al., Proc. Natl. Acad. Sci. USA., 1996, 93, 1759-1763). A receptor of the cell for HCV is CD81, and it has been suggested that the binding is mediated by E2 region of HCV (Pileri P. et al., Science, 1998, 282, 938-941). In addition, it has been shown that the binding to a viral receptor is inhibited not only by an antibody against HVR1, but other region is necessary for the binding. In addition, aggregation of cells is observed due to binding of an antibody against E2 region or CD81 (Flint M. et al., J. Virol., 1999, 73, 6235-6244).

In addition, recently, it has been reported that lactoferrin contained in breast milk binds to HCV and reduces an amount of virus in blood (Tanaka K. et al., Jpn. J. Cancer Res., 1999, 90, 367-371). It has been further reported that a viral region to which lactoferrin binds is E2 region (Akito Nozaki et al., Society of Japan Virology, the 47th academic meeting, general meeting program, abstract, 1999, 188). Further, it has reported that the LDL receptor has an affinity with E2 region [Wunschmann, S. et al., J. Virol., 74, 10055-10062 (2000)].

Since a half-life of virus in blood is deduced to be 2.7 hours (Neumann A U. et al., Science, 1998, 282, 103-107), virus in blood rapidly disappears, when the infection to a cell is suppressed. Standing on this point of view, it is considered that suppression of adhesion of virus to a target cell is effective for eliminating virus in blood in a shorter period of time. However, unless the infected cell is eliminated as described above, complete disappearance of virus cannot be accomplished. Immediately after the administration of interferon, virus in blood is rapidly eliminated, but release of a small amount of virus produces a new infection. Therefore, it is considered that virus is relapsed after the completion of administration of interferon. Therefore, it is considered that a clearance of virus by interferon enhanced by additional administration of a substance which inhibits the infection of virus itself to the administration of interferon having both an anti-virus action and an immuno-stimulating action.

In addition, in the case of HBV, which is an etiologist for hepatitis B, since HBV is a double-stranded DNA virus, it can be hardly considered that a viral genome disappears directly by an anti-virus action of interferon. Also, since HBV is not a cytotoxic virus and an amount of the virus in blood is correlated with a degree of liver dysfunction, it is considered that the liver dysfunction due to HBV is mainly caused by the immunological eliminating action for a viral infected cell. It is considered that an interferon treatment finally eliminates the virus by mainly more effectively enhancing this immunological eliminating action.

HBV is a relatively stable virus. It is considered that if the infection to a target cell can be inhibited, a new infection is protected, whereby the immunological eliminating action in an interferon treatment can be maximally drawn out.

Recently, host proteins binding to pre-S protein of duck hepatitis B virus have been identified. The host proteins were carboxypeptidase D and glycine decarboxylase complex P protein (Tong S. et al., J. Virol., 1999, 73, 8696-8702 and Li J. et al., J. Biol. Chem., 1999, 274, 27658-27665). There has not yet been verified the presence or absence of the affinity of these proteins with pre-S protein in human.

The present inventors have assumed that there is a sequence capable of inhibiting the binding of CD81 as a HCV receptor to HCV in an amino acid sequence of lactoferrin, and thought that the sequence has certain homologies with CD81. In addition, in HBV, the affinity of HBV pre-S to a cell is present in a limited region from 83rd amino acid residue to 107th amino acid residue in the pre-S protein (Tong S. et al., J. Virol., 1999, 73, 8696-8702 and Li J. et al., J. Biol. Chem., 1999, 274, 27658-27665). Therefore, even if there are a plurality of proteins having an affinity to pre-S proteins, the present inventors have deduced that their binding reactions are the same, and thought that there are found some homologies in an amino acid sequence and a tertiary structure in carboxypeptidase D and glycine decarboxylase complex P protein. As a result, the present inventors have found a partially similar sequence from the comparison of an amino acid sequence of lactoferrin, which is thought to protect against the HCV infection, with an amino acid sequence of human CD81, which is a viral receptor, and also found an amino acid sequence which is similar to that of a protein having an affinity to duck HBV pre-S. The present inventors have thought that an oligopeptide sequence or a part thereof, or a compound having a structure similar to tertiary structures of those sequences has been effective for suppressing the viral infection. The present invention has been found based on such a discussion.

The prophylactic or therapeutic agent for chronic hepatitis of the present invention include a prophylactic or therapeutic agent for chronic type-C hepatitis, a prophylactic or therapeutic agent for chronic type-B hepatitis, and the like.

One of the features of the prophylactic or therapeutic agent for chronic type-C hepatitis of the present invention resides in that the prophylactic or therapeutic agent comprises a compound having the following characteristics:

(a) having a binding affinity to an envelop protein of HCV; and

(b) inhibiting binding of HCV to a receptor protein on a target cell for the HCV.

Since the prophylactic or therapeutic agent for chronic type-C hepatitis of the present invention comprises a compound having the above-mentioned characteristics (a) and (b), it is considered that the binding of hepatitis C virus on a target cell is inhibited. Therefore, there are exhibited excellent effects that chronic type-C hepatitis can be prevented or treated.

Concretely, there can be exemplified as the compound having the above-mentioned characteristics (a) and (b), oligopeptides (1) and (2) described below, nucleic acids encoding the oligopeptides, and the like.

(1) Oligopeptide comprising an amino acid sequence or a part thereof, wherein the amino acid sequence is found by identification of a homologous sequence of bovine and human lactoferrins which has been known to bind to HCV, and human CD81, which is a receptor molecule for HCV.

Concretely, there are exemplified oligopeptides shown in the following 1) to 3), which are a region for binding to HCV, the oligopeptides being capable of inhibiting the binding to a viral receptor:

1) an oligopeptide having a part of human-derived CD81 protein (SEQ ID NO: 7), wherein the oligopeptide consists of the amino acid sequence of SEQ ID NO: 1;

2) an oligopeptide having a part of bovine-derived lactoferrin protein (SEQ ID NO: 8), wherein the oligopeptide consists of the amino acid sequence of SEQ ID NO: 2; and

3) an oligopeptide having a part of human-derived lactoferrin protein (SEQ ID NO: 9), wherein the oligopeptide consists of the amino acid sequence of SEQ ID NO: 3.

The nucleic acid encoding an oligopeptide satisfying the above-mentioned requirement 1) includes a nucleic acid comprising a nucleic acid selected from the group consisting of:

(i) a nucleic acid encoding an oligopeptide having the amino acid sequence of SEQ ID NO: 1;

(ii) a nucleic acid having the nucleotide sequence of SEQ ID NO: 4; and

(iii) a part of the nucleic acid encoding an oligopeptide having the amino acid sequence of SEQ ID NO: 1.

In addition, the nucleic acid encoding an oligopeptide satisfying the above-mentioned requirement 2) includes a nucleic acid comprising a nucleic acid selected from the group consisting of:

(iv) a nucleic acid encoding an oligopeptide having the amino acid sequence of SEQ ID NO: 2;

(v) a nucleic acid having the nucleotide sequence of SEQ ID NO: 5; and

(vi) a part of a nucleic acid encoding an oligopeptide consisting of the amino acid sequence of SEQ ID NO: 2.

In addition, the nucleic acid encoding an oligopeptide satisfying the above-mentioned requirement 3) includes a nucleic acid comprising a nucleic acid selected from the group consisting of:

(vii) a nucleic acid encoding an oligopeptide having the amino acid sequence of SEQ ID NO: 3;

(viii) a nucleic acid having the nucleotide sequence of SEQ ID NO: 6; and

(ix) a part of the nucleic acid encoding a polypeptide having the amino acid sequence of SEQ ID NO: 3.

(2) Oligopeptide comprising an amino acid sequence or a part thereof, wherein the amino acid sequence is found by identification of a homologous sequence between a human LDL receptor, which has been known to bind to HCV, and human CD81, which is a receptor molecule for HCV.

Concretely, there are exemplified oligopeptides shown in the following 1′) and 2′), which are a region for binding to HCV, the oligopeptides being capable of inhibiting binding to a viral receptor:

1′) an oligopeptide having a part of a human LDL receptor (SEQ ID NO: 22), wherein the oligopeptide consists of the amino acid sequence of SEQ ID NO: 18; and

2′) an oligopeptide having a part of a human LDL receptor (SEQ ID NO: 22), wherein the oligopeptide consists of the amino acid sequence of SEQ ID NO: 19.

The nucleic acid encoding an oligopeptide satisfying the above-mentioned requirement 1′) includes a nucleic acid comprising a nucleic acid selected from the group consisting of:

(i′) a nucleic acid encoding an oligopeptide having the amino acid sequence of SEQ ID NO: 18;

(ii′) a nucleic acid having the nucleotide sequence of SEQ ID NO: 20; and

(iii′) a part of the nucleic acid encoding an oligopeptide having the amino acid sequence of SEQ ID NO: 18.

In addition, the nucleic acid encoding an oligopeptide satisfying the above-mentioned requirement 2′) includes a nucleic acid comprising a nucleic acid selected from the group consisting of:

(iv′) a nucleic acid encoding an oligopeptide having the amino acid sequence of SEQ ID NO: 19;

(v′) a nucleic acid having the nucleotide sequence of SEQ ID NO: 21; and

(vi′) a part of a nucleic acid encoding an oligopeptide consisting of the amino acid sequence of SEQ ID NO: 19.

The nucleic acid used in the present invention may be a nucleic acid complementary to the nucleic acids of the above-mentioned (i) to (ix) and (i′) to (vi′), and concretely the nucleic acid may be DNA or RNA. In addition, as long as the encoded polypeptides have the above-mentioned characteristics (a) and (b), there can also be used in the present invention (x) a nucleic acid having a nucleotide sequence having substitution, deletion, addition or insertion in at least one base in a nucleotide sequence of a nucleic acid selected from the group consisting of the above-mentioned (i) to (ix); (xi) a nucleic acid capable of hybridizing with a nucleic acid selected from the group consisting of above-mentioned (i) to (x) under the stringent conditions; (vii′) a nucleic acid having a nucleotide sequence having substitution, deletion, addition or insertion in at least one base in a nucleotide sequence of a nucleic acid selected from the group consisting of the above-mentioned (i′) to (vi′); and (viii′) a nucleic acid capable of hybridizing with a nucleic acid selected from the group consisting of the above-mentioned (i′) to (vi′) under the stringent conditions.

The term “stringent conditions” as referred to herein includes conditions described in Sambrook et al., Molecular Cloning: A Laboratory Manual Second eds.,(1980).

Here, each of the nucleotide sequences of SEQ ID NOs: 7 to 9 is a nucleotide sequence of a nucleic acid encoding human-derived protein CD81, a nucleotide sequence of a nucleic acid encoding bovine-derived lactoferrin or a nucleotide sequence of a nucleic acid encoding human-derived lactoferrin. The amino acid sequence of SEQ ID NO: 1 is a part of an amino acid sequence of human-derived CD81 of SEQ ID NO: 7. Also, the amino acid sequence of SEQ ID NO: 2 is a part of an amino acid sequence of bovine-derived lactoferrin of SEQ ID NO: 8. In addition, the amino acid sequence of SEQ ID NO: 3 is a part of an amino acid sequence of human-derived lactoferrin of SEQ ID NO: 9. The nucleotide sequence of SEQ ID NO: 4 is a nucleotide sequence of a nucleic acid encoding an amino acid sequence of human-derived CD81 of SEQ ID NO: 1; the nucleotide sequence of SEQ ID NO: 5 is a nucleotide sequence of a nucleic acid encoding an amino acid sequence of bovine-derived lactoferrin of SEQ ID NO: 2; and the nucleotide sequence of SEQ ID NO: 6 is a nucleotide sequence of a nucleic acid encoding an amino acid of human-derived lactoferrin of SEQ ID NO: 3. Each of SEQ ID NOs: 18 and 19 is a part of an amino acid sequence of human LDL receptor of SEQ ID NO: 22. Each of SEQ ID NOs: 20 and 21 is a nucleotide sequence of a nucleic acid encoding an amino acid sequence of human-derived LDL receptor of SEQ ID NO: 18 or 19.

Further, the compound which can be used in the prophylactic or therapeutic agent for chronic type-C hepatitis of the present invention includes an oligopeptide or a part thereof, wherein the oligopeptide has the following characteristics:

I) consisting of 31 to 33 amino acids which are flanked by cysteine residues;

II) forming a loop structure; and

III) comprising basic amino acid residues at a middle portion and C-terminal side of loop structure;

a cyclic oligopeptide comprising the oligopeptide or a part thereof; and the like.

Concretely, the oligopeptide having the above-mentioned characteristics I) to III) includes a peptide having the amino acid sequence of SEQ ID NO: 1, 2, 3 or 27, and a cyclic peptide having the amino acid sequence of SEQ ID NO: 28.

The oligopeptide used in the present invention can be deduced by comparison with each of an amino acid sequence of bovine lactoferrin, of which binding activity to HCV is known, and an amino acid sequence of CD81.

Concretely, an amino acid sequence is deduced on the basis of each nucleotide sequence of human CD81 (accession No. NM004356) or bovine lactoferrin (accession No. M63502) disclosed in a known database (GenBank), and each amino acid sequence may be compared.

Each peptide is synthesized on the basis of the resulting homologous region, and binding activity of the peptide to a recombinant E2 protein is confirmed. A confirmation of the binding activity can be carried out, for example, by ELISA, immunoprecipitation, RIA or the like.

Thereafter, inhibition of binding of the recombinant E2 protein to a cell expressing CD81, for example, Jurkat cell may be confirmed. The confirmation of inhibition of the binding can be carried out by FACS analysis, or determination of radioactivity bound to a cell in a case where the recombinant E2 protein labeled with a radioactive isotope element is used, besides the above-mentioned ELISA and RIA.

In addition, in order to determine what sort of an amino acid sequence contained in the oligopeptide is effective for suppressing the virus infection, a partial amino acid sequence of the above-mentioned selected sequence is synthesized, whereby selection can be made on the basis of the binding affinity to E2 and the inhibitory effect of binding of E2 to CD81.

One of the features of the prophylactic or therapeutic agent for chronic type-B hepatitis of the present invention resides in that the prophylactic or therapeutic agent comprises a compound having the following characteristics:

(a′) having a binding affinity to S antigen protein of HBV; and

(b′) inhibiting binding of HBV to a receptor protein on a target cell for the HBV.

Since the prophylactic or therapeutic agent for chronic type-B hepatitis of the present invention comprises the compound having the above-mentioned characteristics (a′) and (b′), it is considered that binding of a hepatitis B virus on a target cell is inhibited. Therefore, there are exhibited excellent effects that chronic type-B hepatitis can be prevented or treated.

Concretely, as the compound having the above-mentioned characteristics (a′) and (b′), there are exemplified an oligopeptide described in item (2) below and a nucleic acid encoding the oligopeptide.

(2) Oligopeptide comprising an amino acid sequence or a part thereof, wherein the amino acid sequence is found by identifying a homologous sequence between human carboxypeptidase D and human glycine decarboxylase complex P protein, which are known to bind to HBV

Concretely, there are exemplified oligopeptides shown in the following 1) to 2), which are a region for binding with HBV, the oligopeptides being capable of inhibiting binding to a viral receptor:

1) an oligopeptide having a part of human carboxypeptidase D (SEQ ID NO: 16), wherein the oligopeptide consists of the amino acid sequence of SEQ ID NO: 10; and

2) an oligopeptide having a part of human glycine decarboxylase complex P protein (SEQ ID NO: 17), wherein the oligopeptide consists of the amino acid sequence of SEQ ID NO: 11.

The nucleic acid encoding an oligopeptide satisfying the above-mentioned requirement 1) includes a nucleic acid comprising a nucleic acid selected from the group consisting of:

(I) a nucleic acid encoding an oligopeptide having the amino acid sequence of SEQ ID NO: 10;

(II) a nucleic acid having the nucleotide sequence of SEQ ID NO: 12; and

(III) a part of the nucleic acid encoding an oligopeptide having the amino acid sequence of SEQ ID NO: 10.

The nucleic acid encoding an oligopeptide satisfying the above-mentioned requirement 2) includes a nucleic acid comprising a nucleic acid selected form the group consisting of:

(IV) a nucleic acid encoding an oligopeptide having the amino acid sequence of SEQ ID NO: 11;

(V) a nucleic acid having a nucleotide sequence of SEQ ID NO: 13; and

(VI) a part of the nucleic acid encoding an oligopeptide having the amino acid sequence of SEQ ID NO: 11.

The nucleic acid used in the present invention may be a nucleic acid complementary to the above-mentioned nucleic acids (I) to (VI), and concretely the nucleic acid may be DNA or RNA. In addition, as long as an encoded polypeptide has the above-mentioned characteristics (a′) and (b′), there can be also used in the present invention (VII) a nucleic acid having a nucleotide sequence having substitution, deletion, addition or insertion in at least one base in a nucleotide sequence of a nucleic acid selected from the group consisting of the above-mentioned (I) to (VI), and (VIII) a nucleic acid capable of hybridizing with a nucleic acid selected from the group consisting of the above-mentioned (I) to (VII) under the stringent conditions.

Here, each of the amino acid sequences of SEQ ID NOs: 16 and 17 is an amino acid of human-derived carboxypeptidase D and an amino acid of human-derived glycine decarboxylase complex P protein, respectively. The amino acid sequence of SEQ ID NO: 10 is a part of an amino acid sequence of human-derived carboxypeptidase D of SEQ ID NO: 16. In addition, the amino acid sequence of SEQ ID NO: 11 is a nucleotide sequence of a nucleic acid encoding an amino acid sequence of human-derived glycine decarboxylase complex P protein of SEQ ID NO: 17. The nucleotide sequence of SEQ ID NO: 12 is a nucleotide sequence of a DNA encoding an amino acid sequence of human-derived carboxypeptidase D of SEQ ID NO: 10, and the nucleotide sequence of SEQ ID NO: 13 is a nucleotide sequence of a DNA encoding an amino acid sequence of human-derived glycine decarboxylase complex P protein of SEQ ID NO: 11.

The oligopeptide used in the present invention can be deduced, for example, by comparison with each of amino acid sequences of duck-derived carboxypeptidase D, which is considered to be important for binding to duck HBV, and glycine decarboxylase complex P protein.

Concretely, an amino acid sequence is deduced by nucleotide sequences of duck-derived carboxypeptidase D (accession No. AF039749) and glycine decarboxylase complex P protein (accession No. AF137264) disclosed in a known database (GenBank), and each region associated with binding to duck-derived pre-S may be compared. As to an application to human, a duck-derived sequence is compared with an amino acid sequence deduced from nucleotide sequences of human-derived carboxypeptidase D (accession No. U65090) and glycine decarboxylase complex P protein (accession No. NM000170), and an amino acid sequence of a corresponding human-derived peptide may then be determined.

Each peptide is synthesized on the basis of the obtained homologous region, and the binding affinity of the peptide to the recombinant pre-S protein may be confirmed.

An amino acid sequence of a region effective for suppressing the virus infection in an amino acid sequence contained in the oligopeptide used in the prophylactic or therapeutic agent for chronic hepatitis of the present invention, concretely a prophylactic or therapeutic agent for chronic type-C hepatitis or a prophylactic or therapeutic agent for chronic type-B hepatitis, can be selected by synthesizing a peptide consisting of a part of the amino acid sequence selected as mentioned above and selecting on the bases of the binding activity to a viral antigen and the inhibitory effect of binding of a viral antigen to a virus receptor.

The oligopeptide having the selected amino acid sequence itself may be administered to a patient with chronic type-B hepatitis or chronic type-C hepatitis. Further, the oligopeptide having the above-mentioned selected amino acid sequence is thought to have an enhanced virus clearance ratio by using the oligopeptide in combination with interferon.

In the case of chronic type-C hepatitis, soluble CD81 or lactoferrin itself can be administered. It is preferable that an oligopeptide having only a sequence important for binding to HCV is administered, from the viewpoints of reducing the degradation of a protein absorbed during an oral administration and the side effects during intravenous injection.

The same can be said for the case of chronic type-B hepatitis. The soluble carboxypeptidase D or glycine decarboxylase complex P protein itself can be administered. It is preferable that an oligopeptide having only a sequence important for binding to HBV is administered.

A prophylactic or therapeutic agent comprising as an active ingredient the selected oligopeptide or a part of the oligopeptide, or a nucleic acid encoding thereof can be administered in combination with an adjuvant, or can be administered in a particulate preparation. As a dosage form, more concretely, a prophylactic or therapeutic agent can be prepared into the form of a liposome preparation, a particulate preparation binding to a bead having a diameter of a few μm, a preparation binding to a lipid or the like. The dose can be appropriately adjusted depending upon the properties of an infected cell, age and weight of a patient and the like, and is usually 0.001 mg/kg/dosage to 1000 mg/kg/dosage. It is preferable that the prophylactic or therapeutic agent is administered every day at an initial administration, and thereafter administered once in several days to several months depending upon the amount of virus in a cell. As an administration form, the prophylactic or therapeutic agent can be administered by oral administration, intraarterial injection, intravenous injection, intramuscular injection or local injection to the liver.

In addition, it is preferable that the above-mentioned oligopeptide is stabilized in blood, and the oligopeptide may be modified for that purpose. For example, the oligopeptide can be modified by preparing an oligopeptide stable in blood by connecting the oligopeptide to polyethylene glycol, to lengthen its half-life in blood, or modifying the oligopeptide at N-terminal or substituting with D-amino acid.

According to the above-mentioned oligopeptide, there can be further provided a method for treating chronic hepatitis in combination with the use of interferon. The method for treating chronic hepatitis in combination with the use of interferon is also encompassed within the scope of the present invention. Concretely, one of the characteristics of the above-mentioned method for treatment in combination with the use of interferon resides in that the method uses an oligopeptide or a part thereof in combination with interferon, wherein the oligopeptide having the following characteristics:

(A) having a binding affinity to a viral antigen protein;

(B) inhibiting binding of virus to a receptor protein on a target cell for the virus; and

(C) having homologies with the receptor protein at the amino acid sequence level.

The action of interferon is roughly classified into the anti-virus action directly acting on an infected cell and the immuno-stimulating action of host immunity.

For example, in the case of HCV, the anti-virus action by interferon is generated by signal transmission via an interferon receptor, thereby promoting degradation or translation inhibition of RNA of HCV by inducing production of an anti-virus factor. Usually, in an interferon treatment to chronic type-C hepatitis, since RNA of HCV in serum becomes outside the detection limit two days after the administration at 73.8% of remarkably effective cases, it is considered that HCV is eliminated rapidly after the interferon administration (Saito H. et al., Keio J. Med., 1997, 46, 74-80). However, among these cases in which viruses are disappeared, relapse of virus is found after the termination of the interferon administration in a considerable number of cases. These results mean that even if viral RNA is not detected in blood during the interferon administration, many viruses are present in liver or peripheral blood mononuclear cell (PBMC). In the treatment of chronic type-C hepatitis resistant to the interferon treatment, it is thought that complete elimination of HCV present in an infected cell or immunological elimination of an infected cell is necessary. In order to increase the ratio of virus elimination by interferon, activation of these two actions are to be considered. According to the method for treatment in combination with the use of interferon of the present invention, binding of HCV on a target cell can be inhibited by using the above-mentioned oligopeptide or nucleic acid, so that HCV can be eliminated by interferon, whereby the virus clearance ability can be made more effective after the start of the interferon administration. As a result, relapse of interferon-resistant virus at the termination of the interferon administration can be further reduced.

In the present invention, the selected oligopeptide can be fused with another functional molecule to be used as a fusion substance. The fusion substance is also encompassed in the present invention.

In this case, it is preferable that a site into which an oligopeptide sequence is inserted is a position at which the activity of the functional molecule is not reduced. In particular, fusion of an interferon molecule and a selected oligopeptide sequence is advantageous in the interferon treatment in that a single dose administration can be made.

Concretely, the fusion interferon includes a fusion interferon comprising an amino acid sequence of an oligopeptide having the following characteristics:

(A) having a binding affinity to a viral antigen protein;

(B) inhibiting binding of virus to a receptor protein on a target cell for the virus; and (C) having homologies with the receptor protein at the amino acid sequence level.

More specifically, there are included a fusion interferon comprising at least one kind of an amino acid sequence or a part thereof, wherein the amino acid sequence is selected from the group consisting of SEQ ID NOs: 1, 2 and 3; a fusion interferon comprising an amino acid sequence of SEQ ID NO: 10 or 11 or a part thereof.

When an oligopeptide sequence is inserted into an interferon molecule, the corresponding oligopeptide may be conveniently connected to N-terminal or C-terminal of interferon. Since a neighbor region of N-terminal or C-terminal of interferon is not a region essential for binding of interferon to the interferon receptor, it is determined that interferon retains the activity with a high possibility by addition of the oligopeptide sequence to the terminal. However, a structure of a connected amino acid sequence influences the interferon activity in some cases. In that case, an oligopeptide sequence which does not lower the activity of interferon and can bind to a virus antigen may be limited.

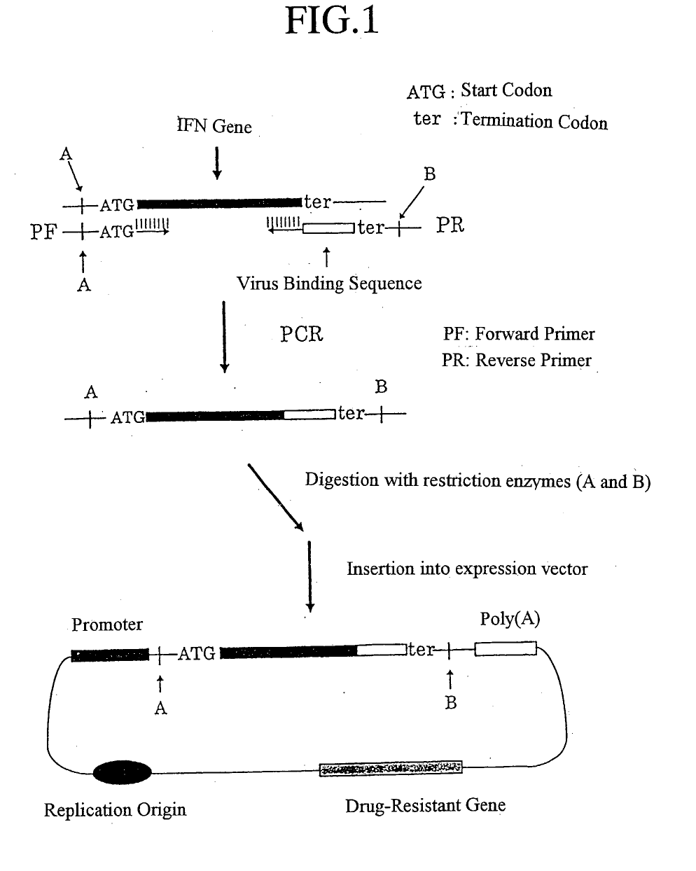

In this case, an interferon gene may be amplified using PCR primers comprising a region having homologies with a 5′-terminal side or a 3′-terminal side of a DNA encoding the corresponding oligopeptide and a DNA encoding interferon. In addition, it is desired that to add a restriction enzyme cleavage site to a 5′-side of a primer for insertion into an expression vector, because insertion into an expression vector is conveniently carried out. An example of the construction of an interferon gene with the corresponding oligopeptide added on C-terminal side is shown in FIG. 1. Hereinafter, the interferon with the corresponding oligopeptide added on C-terminal is referred to as “C-terminal added interferon,” and the interferon with the corresponding oligopeptide added on N-terminal side is referred to as “N-terminal added interferon.”

In the production of C-terminal added and N-terminal added interferons, any cell strains derived from prokaryote or eukaryote can be used as a host. In the case of C-terminal added interferon, a termination codon is incorporated at 3′-terminal of a DNA encoding an oligopeptide to be added on C-terminal side. On the other hand, in the case of N-terminal added interferon, when the prokaryote is used as a host, a primer may be designed so that a DNA sequence encoding an oligopeptide having an initiation codon at 5′-terminal is contained. On the other hand, when a cell strain derived from eukaryote is used as a host, a primer may be designed so that an amino acid sequence of an oligopeptide is inserted between C-terminal of a signal sequence of interferon and N-terminal of processed interferon.

The original interferon gene used as a template in reaction for PCR may be any of interferon-α, interferon-β, and interferon-γ genes. When an interferon-α gene having many subtypes is used, a gene can be cloned by RT-PCR method from Namalwa cell and the like which have been stimulated and infected with NDV or Sendai virus in accordance with the nucleotide sequences described in the database.

The usefulness of C-terminal added and N-terminal added interferons is as follows:

1) by binding of C-terminal added or N-terminal added interferon to an envelope protein of HCV or S antigen of HBV, the adhesion of HCV or HBV to a cell can be inhibited; therefore, the anti-virus action can be dramatically enhanced because of rapid occurrence of clearance of HCV or HBV from blood;

2) it is anticipated that a binding constant of an oligopeptide added to HCV and interferon is not so high; also, even if a cell is infected via HCV E2 antigen to which C-terminal added and N-terminal added interferons are not bound, C-terminal added and N-terminal added interferons are present in a large amount in the surrounding of an infected cell; since C-terminal added and N-terminal added interferons have far higher affinity to an interferon receptor than to virus, enhancement of the local interferon action can be expected; and

3) by using C-terminal added and N-terminal added interferons bound to HCV or HBV, even if interferon disappears from blood after the administration, the interferon action easily sustains as long as virus bound to C-terminal added and N-terminal added interferons exist.

In addition, although it is considered that interferon with its terminal modified easily retains the original activity, there is a possibility that a new antigenicity becomes problematic. There is a possibility that the antigenicity is produced due to an amino acid sequence for a part for connecting interferon with an added oligopeptide. However, in the tertiary structure, it is deduced that addition of an oligopeptide to a terminal does not produce a great change in a tertiary structure. Therefore, since an added sequence has extremely high homologies with a human sequence, it is deduced that the antigenicity of a sequence itself is extremely low.

The fusion interferon of the present invention can be applied to any virus as long as a disease is a viral disease with which the interferon exhibits an anti-virus activity, in addition to human hepatitis C or B virus disease. Also, the fusion interferon can be applied to mammals such as ducks, chickens, horses, cows, sheep, dogs, cats and the like besides human.

The present invention will be described in further detail by means of examples, without intending to limit the present invention thereto.

### EXAMPLE 1

Analysis of Homologous Region

(1) Studies on Homologous Region of Human CD81, and Human and Bovine Lactoferrins

Based on nucleotide sequences of human CD81 (accession No. NM004356), bovine lactoferrin (accession No. M63502), and human lactoferrin (accession No. NM002343) genes, amino acid sequences were deduced from open reading frames contained therein. Each of the amino acid sequences is shown in SEQ ID NOs: 7 to 9.

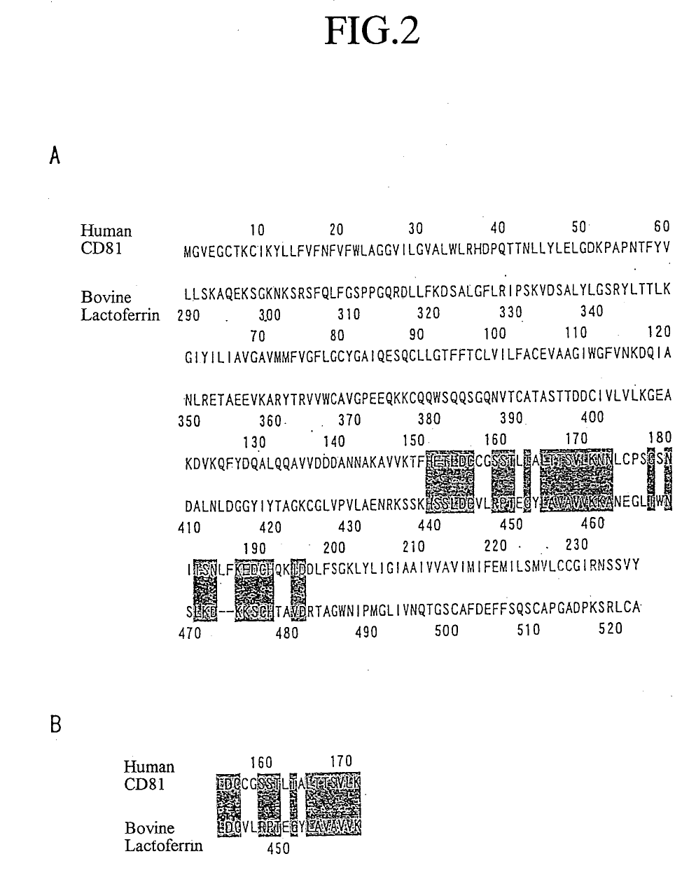

The amino acid sequence homologies between human CD81 and bovine lactoferrin were obtained. As a result, the homologies were found between a region of 151st amino acid residue to 195th amino acid residue of CD81 (amino acid NOs: 151 to 195 of SEQ ID NO: 7) and a region of 439th amino acid residue to 481st amino acid residue of bovine lactoferrin (amino acid NOs: 439 to 481 of SEQ ID NO: 8). A region for which the homologies were found is shown in FIG. 2. In FIG. 2, Panel A of the sequence shows human-derived CD81 and Panel B shows bovine lactoferrin.

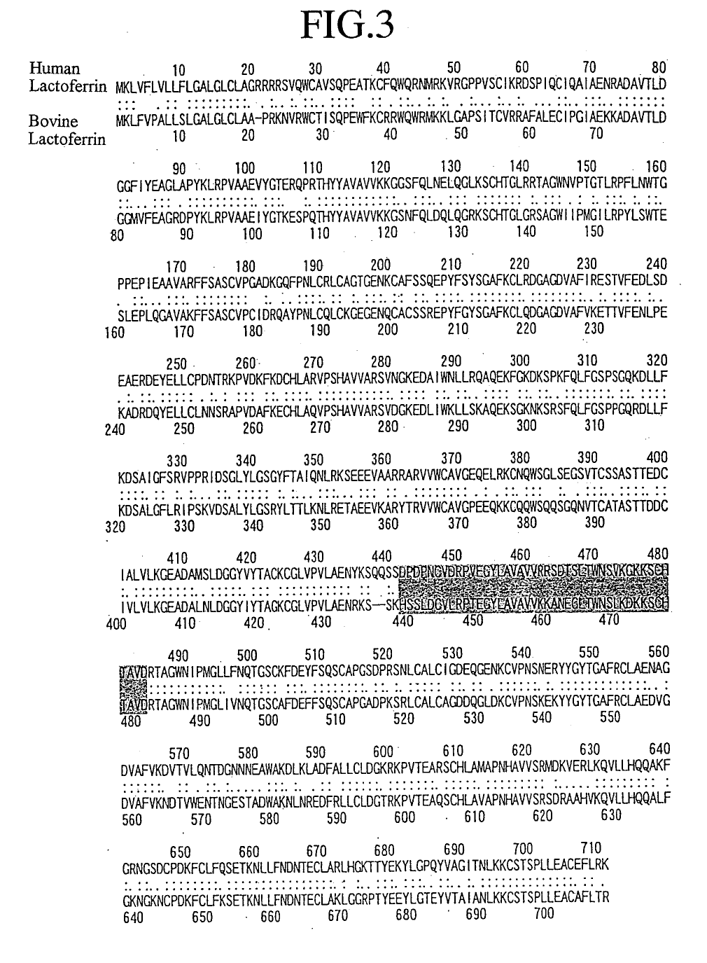

In addition, the homologies of amino acid sequences between bovine lactoferrin and human lactoferrin were studied. As a result, it was clarified that the sequence of 439th amino acid residue to 481st amino acid residue of bovine lactoferrin (amino acid NOs: 439 to 481 of SEQ ID NO: 8) corresponded to a sequence of 442nd amino acid residue to 484th amino acid residue of human lactoferrin (amino acid NOs: 442 to 484 of SEQ ID NO: 9), showing the high homologies between bovine-derived lactoferrin and human-derived lactoferrin. The analytical results on a homologous region are shown in FIG. 3. In the sequence of FIG. 3, an upper row shows bovine-derived lactoferrin, and a lower row shows human-derived lactoferrin. In the figure, a region for which the homologies with CD81 were exhibited is indicated.

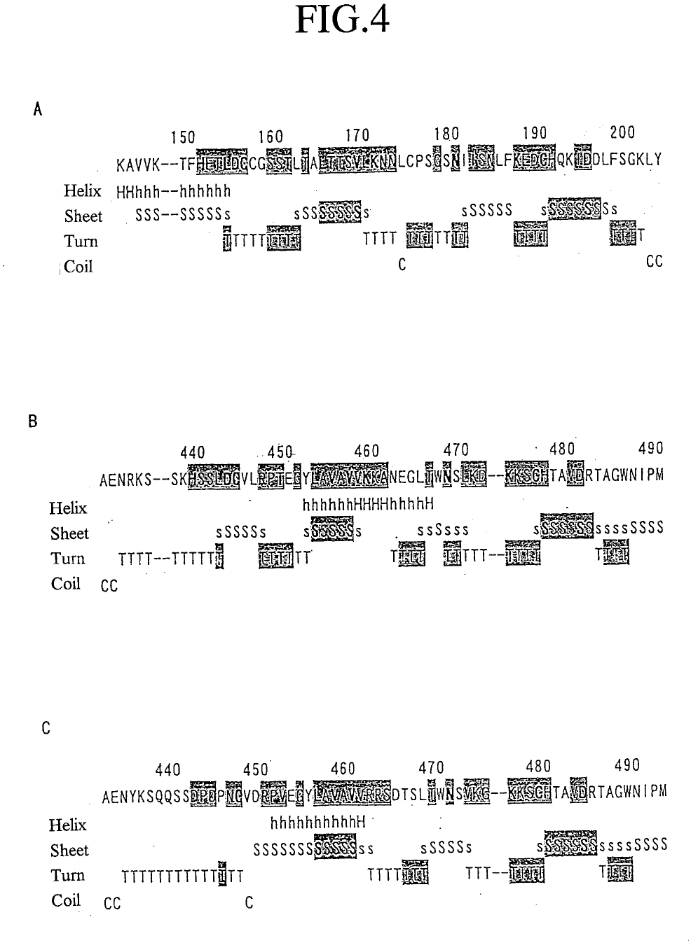

A secondary structure of a partial amino acid sequence of each protein to which HCV is considered to bind was predicted. The results were shown in FIG. 4. The homologous sequences between human CD81 and bovine lactoferrin are indicated in the figure. In the homologous sequences, the identical amino acids were indicated by bold characters, and the homologous amino acids were indicated by plain characters. In addition, in the prediction of the secondary structure, portions corresponding to amino acids constituting the same secondary structure between human CD81 and bovine lactoferrin were shaded with meshed patterns. In the case of human lactoferrin, the same portions as amino acids keeping a common secondary structure between a homologous sequence with human CD81, human CD81 and bovine lactoferrin in the amino acid sequence were shaded with meshed patterns.

As shown in FIG. 4, it was found that the three sequences have the extremely similar structures except that in a homologous region, a structure of 450th amino acid residue to 453rd amino acid residue of human lactoferrin (amino acid NOs: 450 to 453 of SEQ ID NO: 9) takes Sheet structure, while the structures of those in human CD81 and bovine lactoferrin take Turn structure.

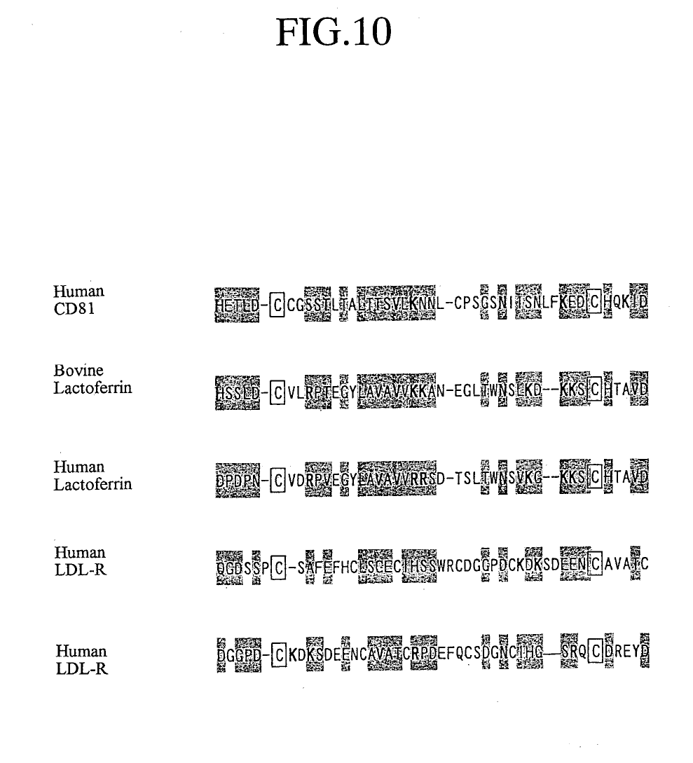

In addition, as shown in FIG. 10, a sequence of human CD81 and sequences of bovine and human lactoferrins were compared. As a result, it was clarified that two cysteine residues existed in similar positions. The cysteine residues were positioned such that 33 amino acids were flanked by the cysteine residues in human CD81, and the cysteine residues were positioned such that 33 amino acids were flanked by the cysteine residues in bovine and human lactoferrins. This means that these regions have a common tertiary structure. It is considered that loop structure is kept by disulfide bond between the cysteine residues. Further, there was found the presence of basic amino acids consisting of arginine and lysine at a middle part of this loop structure and in the neighborhood of cysteine residue on C-terminal side. It is considered that the above-mentioned structural motif is important for binding of HCV. This shows that forming loop structure can increase the binding activity for virus by 1) forcibly generating disulfide bond in an oligopeptide, or 2) connecting N-terminal and C-terminal of an oligopeptide with a compound.

(2) Studies on Homologous Region Between Human CD81 and LDL Receptor

Based on a nucleotide sequence of a human LDL receptor (accession No. NM000527) gene, an amino acid sequence was deduced from an open reading frame existing in the above-mentioned gene. An amino acid sequence excluding a signal sequence is shown in SEQ ID NO: 22.

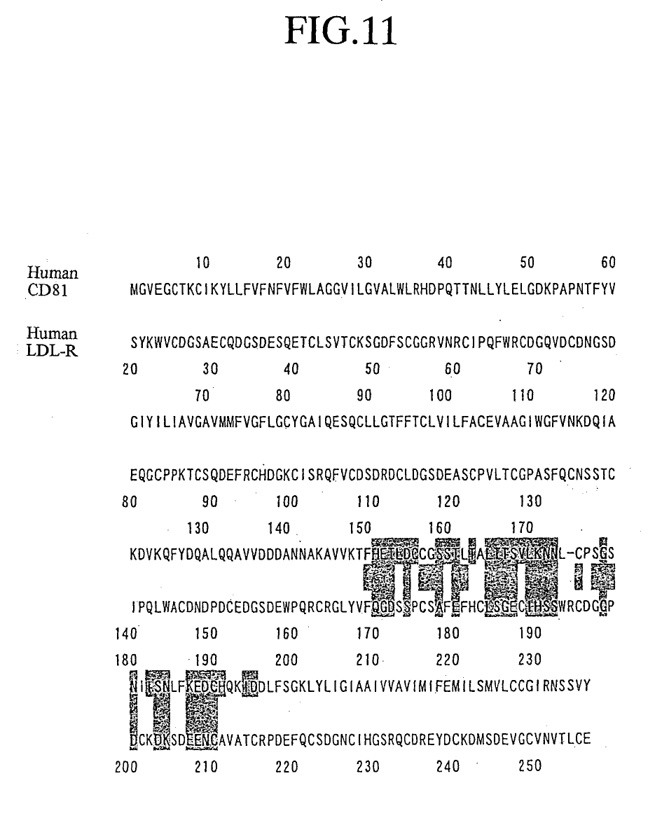

The amino acid sequence homologies between human CD81 and human LDL receptor were obtained. As a result, the homologies were found between a region of 150th amino acid residue to 191st amino acid residue of human CD81 (amino acid NOs: 150 to 191 of SEQ ID NO: 7) and a region of 170th amino acid residue to 210th amino acid residue of a human LDL receptor (amino acid NOs: 170 to 210 of SEQ ID NO: 22). A region for which the homologies were found is shown in FIG. 11. In the sequence of FIG. 11, an upper row shows human-derived CD81, and a lower row shows a human LDL receptor. Portions shaded with meshed patterns in the amino acid sequence of CD81 are amino acid residues having the homologies with bovine and human lactoferrins, and portions shaded with meshed patterns of the amino acid sequence of a human LDL receptor are regions where the homologies were conserved.

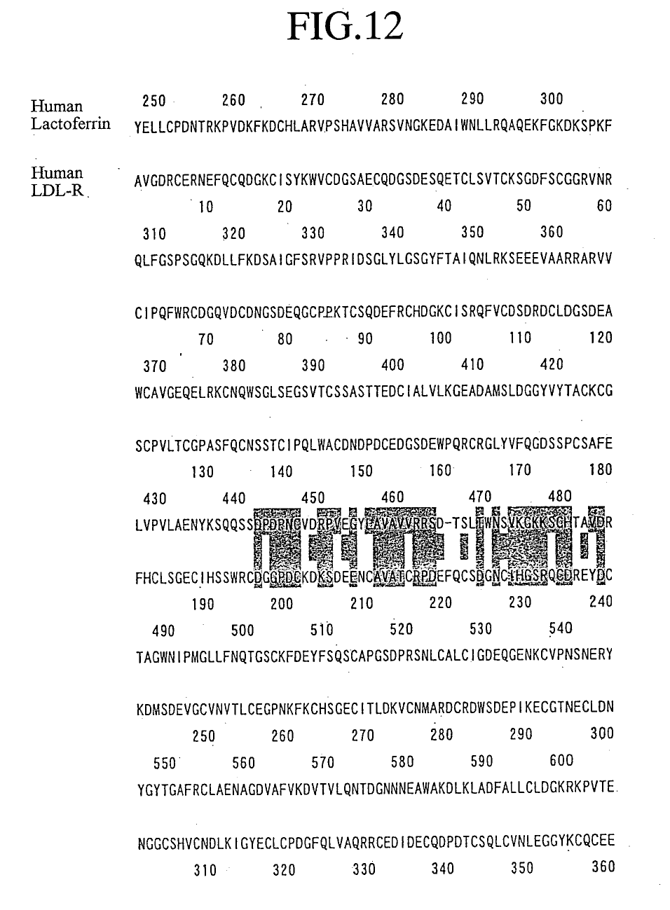

In addition, the homologies between an amino acid sequence of human lactoferrin and that of a human LDL receptor were studied. As a result, the homologies were found between a region of 442nd amino acid residue to 484th amino acid residue of human lactoferrin (amino acid NOs: 442 to 484 of SEQ ID NO: 9) and a region of 196th residue to 239th residue of a human LDL receptor (amino acid NOs: 170 to 210 of SEQ ID NO: 22). A region for which the homologies were found is shown in FIG. 12. As to the amino acid sequences shaded with meshed patterns, they are as defined in FIG. 11.

The results of comparison of selected two kinds of human LDL receptor sequences and a homologous region between human CD81, and bovine and human lactoferrins are shown in FIG. 10. As shown in FIG. 10, it can be seen that cysteine residues in both sequences of two kinds of human LDL receptor sequences exist at similar positions with those of human CD81, and bovine and human lactoferrins. In addition, a region flanked by the cysteine residues was studied. As a result, it is shown that a region having the homologies with the human lactoferrin sequence is more consistent with a motif. However, since basic amino acids also exist in the neighborhood of cysteine residues on C-terminal side in the sequence selected by the homologies with human CD81, possibilities of the selected sequence to bind are suggested.

### EXAMPLE 2

Studies on Binding Affinity to HCV E2 Region

An oligopeptide is synthesized in accordance with each amino acid sequence of homologous regions of human CD81, bovine lactoferrin and human lactoferrin. The resulting oligopeptide is coated on an ELISA plate in accordance with a conventional method. Next, a binding reaction is carried out by adding thereto HCV E2 antigen of Austral Biologicals, Inc. which can be purchased from Cosmo Bio.

As a control, an ELISA plate is coated with bovine lactoferrin protein of SERVA Electrophoresis GmbH. which can be purchased from Cosmo Bio, or human lactoferrin protein of Cappel Product.

After binding with the E2 protein and washing, an E2 antigen bound to an oligopeptide coated on a plate is detected by using a monoclonal antibody for the HCV E2 antigen of Austral Biologicals, Inc. which can be purchased from Cosmo Bio.

### EXAMPLE 3

Selection of Amino Acid Sequence Binding to HCV

An oligopeptide consisting of about 20 amino acids is synthesized so as to overlap with the oligopeptide sequence of which binding activity to HCV E2 antigen in Example 2 is found. In accordance with Example 2, a region having the highest binding activity to the HCV E2 antigen may be selected, and an binding sequence may be further narrowed down. A binding experiment is carried out in the same manner, whereby a smallest oligonucleotide sequence which can bind to HCV E2 antigen can be identified.

### EXAMPLE 4

Studies on Inhibition of Binding of CD81 to HCV

An oligopeptide capable of binding to HCV E2 antigen is reacted with the HCV E2 antigen to study whether or not binding of CD81 expressed in Jurkat cells purchased from DAINIPPON PHARMACEUTICAL CO., LTD. to the HCV E2 antigen is inhibited. By the study, whether or not the selected oligopeptide inhibits adhesion of the HCV E2 antigen to CD81-positive cells can be estimated. The inhibitory activity for adhesion of CD81 to the IICV E2 antigen was assayed in accordance with the literature. The concrete description will be given hereinbelow.

(1) Confirmation of CD81 Expression Using FACScan Jurkat cells (ATCC TlB-152) collected from a culturing T-75 flask (Coaster) in accordance with the conventional method were washed with centrifugation twice with 10 mL of Wash Buffer [phosphate-buffered saline (manufactured by Lifetech Oriental) containing 1% fetal bovine serum (manufactured by BIO WHITTAKER) and 0.1% sodium azide (manufactured by nakalai tesque); hereinafter simply referred to as WB].

Washed Jurkat cells were suspended in WB so as to have a concentration of 4×10⁶ cells/mL to give a Jurkat cell suspension. Twenty microliters of an anti-human CD81 antibody (BD PharMingen) was added to a 6 mm round-bottom tube (manufactured by Falcon). Further, 50 μL of the above-mentioned Jurkat cell suspension was added thereto, to give an anti-human CD81 antibody-Jurkat cell mixture.

In order to confirm the specificity of staining, 1 μg of a mouse IgG1 antibody (manufactured by BD PharMingen), the same isotype as the anti-human CD81 antibody, was added to a 6 mm round-bottom tube (manufactured by Falcon). Further, 50 μL of the above-mentioned Jurkat cell suspension was added thereto, to give a mouse IgG1 antibody-Jurkat cell mixture.

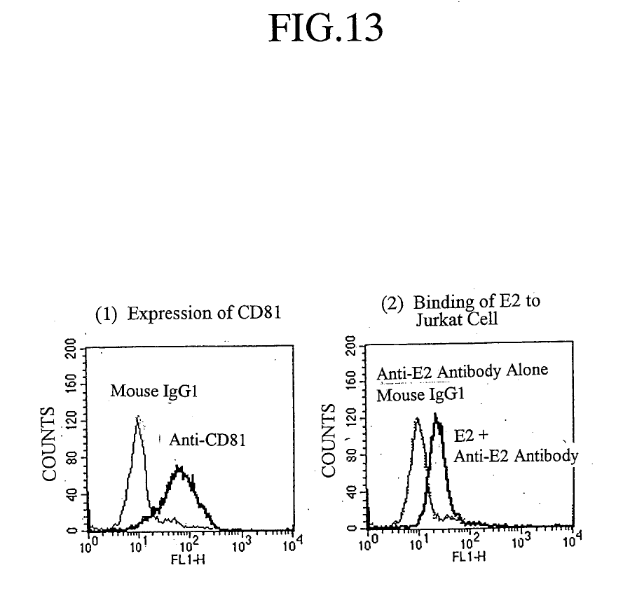

Each of the resulting anti-human CD81 antibody-Jurkat cell mixture and the mouse IgG1 antibody-Jurkat cell mixture was stirred, and each of these mixtures were then allowed to stand on ice for 30 minutes. Thereafter, both mixtures were washed with centrifugation twice with 2 mL of WB, and thereafter suspended in 0.1 mL of WB containing 1 μg of the FITC-labeled anti-mouse IgG1 antibody (manufactured by BD PharMingen). Each of the resulting suspensions was stirred, and thereafter allowed to stand on ice for 30 minutes. Subsequently, the resulting product was washed with centrifugation twice with 2 mL of WB, and thereafter suspended in 0.2 mL of a 10% neutral buffered formalin solution (manufactured by nakalai tesque). The resulting aliquots were analyzed by using FACScan (manufactured by Nihon Becton Dickinson). The above results are shown in FIG. 13. In Panel (1) of FIG. 13, when stained with the anti-human CD81 antibody, the FITC fluorescent intensity became stronger, as compared to that stained with the mouse IgG1. The results show that CD81 is expressed on the surface of the Jurkat cells. On the other hand, in Panel (2), the FITC fluorescent intensity was increased only when the Jurkat cells which had been reacted with the recombinant E2 protein, were stained with the anti-E2 antibody. Therefore, it is shown that the recombinant E2 protein is bound to the Jurkat cells.

As a result, as shown in FIG. 13, it can be seen that CD81, which is a receptor for HCV E2 protein, is expressed on almost all the Jurkat cells.

(2) Analysis on Binding of Recombinant E2 Protein to Jurkat Cells

The Jurkat cells were prepared in the same manner as in item (1), and suspended in WB so as to have a concentration of 4×10⁶ cells/mL, to give a Jurkat cell suspension. One microgram of the recombinant E2 protein (Austral Biologicals) was added to a 6 mm rounded-bottom tube, and 50 μL of the Jurkat cell suspension was added thereto, to give a recombinant E2 protein-Jurkat cell mixture.

The above-mentioned recombinant E2 protein-Jurkat cell mixture was stirred and thereafter allowed to stand on ice for 30 minutes. The above-mentioned mixture was washed with centrifugation twice with 2 mL of WB, and thereafter suspended in 0.1 mL of WB containing 1 μg of an anti-E2 antibody (Austral Biologicals). After stirring, the mixture was allowed to stand on ice for 30 minutes.

Hereinafter, in the same manner as in the item (1) above, the resulting product was reacted with an FITC-labeled anti-mouse IgG1 antibody, and thereafter the reaction mixture was suspended in 0.2 mL of a 10% neutral buffered formalin solution. As to the resulting product, E2 protein bound to Jurkat cells was analyzed by using FACScan. The results are shown in FIG. 13.

The FITC fluorescent intensity which had been increased by the addition of the untreated recombinant E2 protein to the Jurkat cells was weakened by the reaction of a peptide or protein with the recombinant E2 protein. This shows that the peptide or the protein inhibited the binding activity of the recombinant E2 protein to the Jurkat cells.

Accordingly, as shown in FIG. 13, it can be seen that the E2 protein is bound to the Jurkat cells.

(3) Peptide Synthesis

Peptides having amino acid sequences of SEQ ID NOs: 23 to 28 were synthesized on the solid phase by Fmoc method, and purified by reverse phase HPLC. Each of the amino acid sequences shows the following peptides:

SEQ ID NO: 23 Human CD81-derived peptide: TAPA148,

SEQ ID NO: 24 Bovine lactoferrin-derived peptide: bLF443,

SEQ ID NO: 25 Human lactoferrin-derived peptide: hLF445,

SEQ ID NO: 26 Human LDL receptor-derived peptide: hLDLR194,

SEQ ID NO: 27 Human LDL receptor-derived peptide: hLDLR220,

SEQ ID NO: 28 Bovine lactoferrin-derived cyclic peptide: cbLF445.

Cyclization of the above-mentioned bovine lactoferrin-derived cyclic peptide of SEQ ID NO: 28 was carried out by forming a disulfide bond with cysteine residues positioned at N-terminal and C-terminal. Further, the cyclization was confirmed by mass spectroscopy of the purified peptide.

(4) Preparation of Peptide Solution

The peptide obtained in the item (3) above was dissolved in a phosphate buffered saline (hereinafter, referred to as PBS; manufactured by Nissuiseiyaku) so as to have a concentration of 1 mg/mL. In addition, bovine lactoferrin (SERVA Electrophoresis GmbH) was dissolved in PBS so as to have a concentration of 1 mg/mL. Thereafter, each of the resulting solutions was sterilized by filtration using a 0.2 μm cartridge filter (Millipore Corporation). Each of the resulting filtrates was dispensed in an amount of 0.2 mL of and frozen-stored at 80° C. The resulting solutions are referred to as a peptide solution and a purified bovine lactoferrin solution.

(5) Studies on Inhibitory Action of Binding of E2 by Peptide

One microgram of the recombinant E2 protein, and 10 μL of the TAPA148, bLF or bLF443 solution obtained in item (4) above were added to a 6 mm rounded-bottom tube and, and WB was then added thereto, to give 50 μL of a mixture. The resulting mixture was stirred and allowed to stand at room temperature for 1 hour. Fifty microliters of the Jurkat cell suspension (4×10⁶ cells/mL) described in item (1) above was added to the resulting solution, and the mixture was stirred and then allowed to stand on ice for 30 minutes. Hereinafter, in the same manner as in item (2) above, the resulting solution was reacted successively with an anti-E2 antibody and an FITC-labeled anti-mouse IgG1 antibody, and the reaction mixture was suspended in 0.2 mL of a 10% neutral buffered formalin solution. As to the resulting suspension, E2 protein bound to Jurkat cells was analyzed by using FACScan.

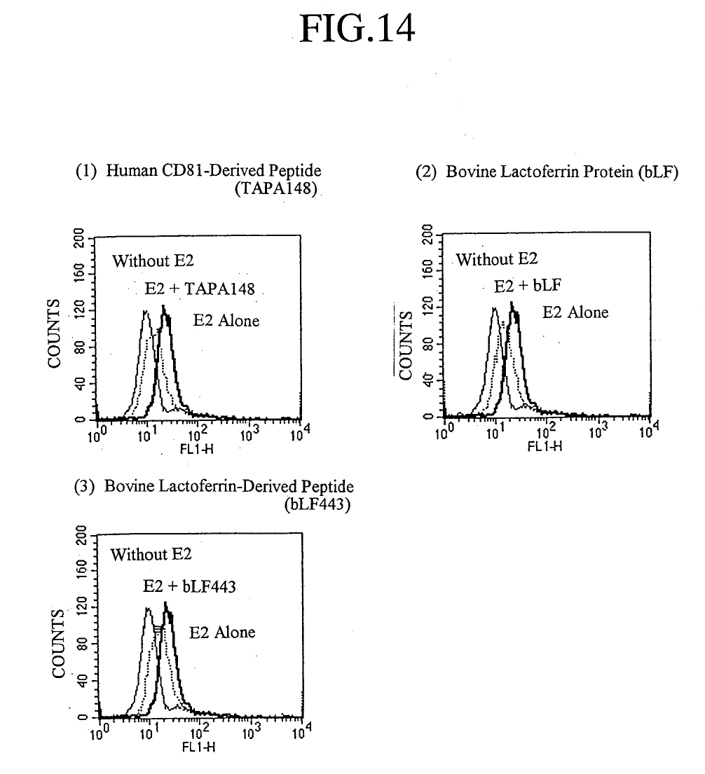

As shown in FIG. 14, there is found the inhibitory action of binding of E2 protein by the human CD81-derived peptide (TAPA148). In addition, there is likewise found the inhibitory action of binding of the E2 protein by the bovine lactoferrin (bLF). Further, there is likewise found the inhibitory action of binding of the E2 protein when the bovine lactoferrin-derived peptide (bLF443) is used.

### EXAMPLE 5

Preparation of Interferon to Which HCV-Bound Oligopeptide is Added

A complementary nucleotide sequence having about 20 bases, immediately before a termination codon of interferon-α2b, and a DNA encoding the selected oligopeptide are fused, and a termination codon sequence was further connected to 3′-terminal, and there are further synthesized 3′-side PCR primers having a restriction enzyme digesting sequence so that the sequence can be cloned into an appropriate restriction enzyme site of pBK-CMV, which can be purchased from Stratagene. In addition, there is synthesized a DNA having a restriction enzyme digesting sequence at a 5′-terminal as a 5′-side primer, and containing a 5′-upstream region of interferon-α2b and a homologous nucleotide sequence having about 20 bases. Thereafter, reaction of PCR is carried out by using a cDNA clone of interferon-α2b obtained with Namalwa cells which can be purchased from DAINIPPON PHARMACEUTICAL CO., LTD. as a template. The resulting PCR amplified fragment is digested with a restriction enzyme, and then inserted into pBK-CMV, to give an Escherichia coli transformant in accordance with a conventional method. A recombinant plasmid harboring the above-mentioned PCR-amplified fragment is isolated from the resulting transformant. Next, appropriate cells (for example, COS-1 cells, CHO cells etc.) are transfected with the resulting recombinant plasmid by calcium phosphate method, to give C-terminal added interferon-α2b. The resulting culture supernatant is added to various human-derived cell strains (HeLa cells, HepG2 cells and the like), whereby confirming that 2-5A synthase is induced by C-terminal added interferon-α2b. In addition, the resulting C-terminal added interferon-α2b and HCV E2 antigen are incubated, and it is confirmed that adhesion of CD81 expressed in the Jurkat cells to HCV E2 antigen is inhibited by C-terminal added interferon-α2b in accordance with the method described in Example 4. The concrete description will be given hereinbelow.

It is noted that, in the present experiment, the C-terminal added interferon-α2b was produced using COS-1 cells (purchased from The Institute of Physical and Chemical Research).

(1) Cloning of Human Interferon-α2b Gene by PCR and Preparation of Interferon-α2b Expression Vector

PCR was carried out by using KOD Plus DNA polymerase (TOYOBO CO., LTD.) and interferon-α2b-specific primers U-2 (SEQ ID NO: 29) and L-2 (SEQ ID NO: 30), with human genome DNA (manufactured by Promega; cat#: G304A, lot#: 9273102L) as a template. In PCR, there was used the following reaction solution [a solution prepared by mixing 988 ng of human genome DNA, 10 μl of KOD Plus DNA polymerase 10× reaction buffer, 10 μl of dNTPs (2 mM each), 4.8 μl of MgSO₄ (25 mM), 5 μl of primer U-2 (0.1 μg/μl), 5 μl of primer L-2 (0.1 μg/μl) and 2 μl of KOD Plus DNA polymerase (1 U/μl), and adding distilled water thereto to make up a volume of 100 μl].

As a thermal cycler, GeneAmp 9700 (manufactured by Perkin-Elmer) was used. The thermal profile comprises 1 cycle of 95° C.-3 minutes, 30 cycles of (95° C.-20 seconds, 60° C.-30 seconds, and 72° C.-3 minutes), and 1 cycle of 72° C.-7 minutes, and thereafter, the reaction mixture was allowed to stand at 4° C. overnight.

The resulting PCR product was purified using QIAGEN PCR Purification Kit in accordance with the attached instruction. The purified DNA fragment was digested with BamHI and HindIII, and the resulting product was purified. In addition, pcDNA3.1(−) (manufactured by Invitrogen) was digested with BamHI and HindIII, and the resulting product was purified. A DNA fragment was inserted into BamHI and HindIII site of the resulting pcDNA3.1(−) digest. By the above procedures, the IFN-α2b expression vector (plasmid was named pCIA) was prepared. In addition, the nucleotide sequence was read and compared with GenBank accession #J00207, V00544, and confirmed to be correct.

(2) Preparation of HCV-Binding type IFNs Expression Vectors

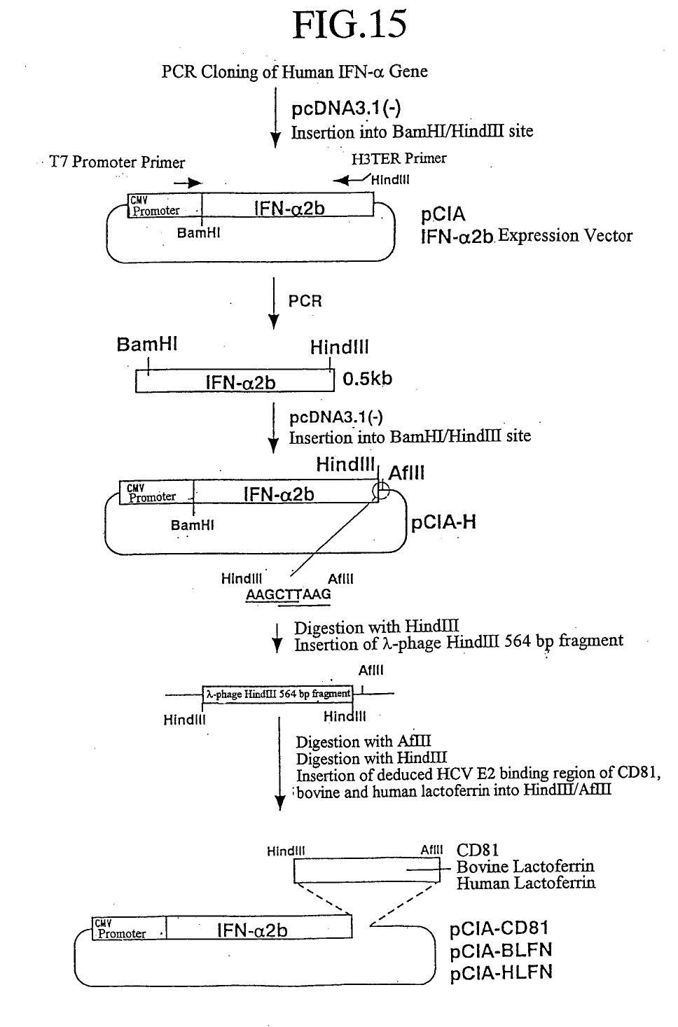

An expression vector for a protein, in which human CD81, bovine lactoferrin or a deduced HCV E2 binding region is fused to C-terminal of human IFN-α2b (hereinafter, referred to as “HCV-binding type IFNs”) was prepared by the following procedures 1), 2) and 3) (FIG. 15).

1) Introduction of HindIII Site as Connecting Site into C-Terminal of IFN-α2b by PCR

PCR was carried out by using T7 promoter primer and H3TER primer (SEQ ID NO: 31) with the IFN-α2b expression vector pCIA as a template, to give a DNA fragment in which HindIII site is introduced into a nucleotide sequence corresponding to C-terminal of IFN-α2b, and thereafter the DNA fragment was inserted into pcDNA3.1(')(plasmid was named pCIA-H). The HindIII site was made so that an amino acid sequence of IFN-α2b was not changed.

2) Preparation of Deduced HCV E2 Binding Regions of Each of CD81, Bovine Lactoferrin and Human Lactoferrin by Synthetic DNA

A synthetic DNA of a deduced HCV E2 binding region of each of CD81, bovine lactoferrin and human lactoferrin was prepared, and the DNA was cloned into pUC18. The deduced HCV E2 binding region of CD81 is shown in SEQ ID NO: 32; the deduced HCV E2 binding region of bovine lactoferrin is shown in SEQ ID NO: 33; and the deduced HCV E2 binding region of human lactoferrin is shown in SEQ ID NO: 34. A nucleotide sequence of each of the resulting clones was confirmed by sequencing.

3) Ligation of IFN-α2b Gene with Each of Deduced HCV E2 Binding Regions of CD81, Bovine Lactoferrin and Human Lactoferrin

There was prepared a vector in which a HindIII fragment having 564 bp of γ phage as a stuffer sequence is once inserted into HindIII site of pCIA-H. The resulting vector was digested with AfIII and then digested with HindIII. Into the resulting product was inserted each of the DNA fragments encoding deduced HCV E2 binding regions of CD81, bovine lactoferrin and human lactoferrin, the DNA fragments resulting from digestion from the plasmid prepared in item 2) above with HindIII and AfIII. A nucleotide sequence of each of the resulting clones was confirmed by sequencing.

By the above procedures, there were prepared expression vectors (HCV-binding type IFN expression vectors) pCIA-CD81, pCIA-BLFN and pCIA-HLFN, resulting from fusion of each of deduced HCV E2 binding regions of human CD81, bovine lactoferrin and human lactoferrin to C-terminal of human IFN-α2b.

(3) Protein Synthesis by in vitro Transcription/Translation and Western Blot Analysis of IFN-α

Without using a radioactive isotope, protein synthesis was carried out by in vitro transcription/translation using TNT T7 coupled wheat germ extract system (manufactured by Promega), with pCIA, pCIA-CD81, pCIA-BLFN and pCIA-HLFN as a template. The resulting products were subjected to 15% SDS-PAGE. Thereafter, the proteins in the gel after electrophoresis were transferred to a membrane (Immobillon P, manufactured by Millipore Corporation). Western blot analysis was carried out by using the resulting membrane and an anti-IFN-α monoclonal antibody (clone MMHA-2, manufactured by PBL Biomedical). Here, the blocking in Western blot was carried out by using a blocking buffer [TBS-T (composition: 50 mM Tris-HCl (pH 7.5), 15 mM NaCl, 0.05% Tween 20) containing 3% normal goat serum (Vector)]. In addition, a dilution prepared by 1000-fold-dilution of the anti-IFN-α t monoclonal antibody with a blocking buffer was used as a primary antibody, and a dilution obtained by 5000-fold-dilution of a peroxidase-labeled anti-mouse IgG goat antibody (manufactured by BIOSOURCE INTERNATIONAL) with a blocking buffer was used as a secondary antibody. Detection was made by using ECL Plus (manufactured by Amersham Pharmacia).

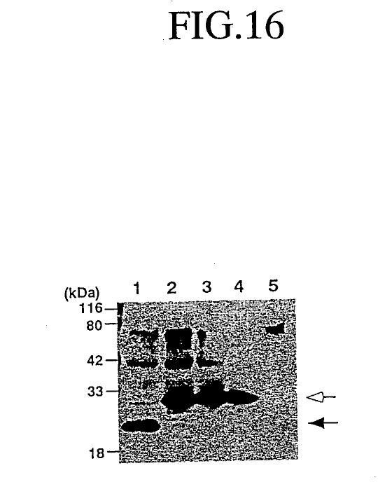

As shown in FIG. 16, it can be seen that IFN-α2b and HCV-binding type IFNs are synthesized with each plasmid as a template.

(4) Transient Expression in COS-1 Cells

COS-1 cells were transfected with each of pCIA, pCIA-CD81, pCIA-BLFN and pCIA-HLFN by using FuGENE6 (Roche Diagnostics GmbH). The culture after the transfection was carried out on DMEM medium without addition of an antibiotic and without addition of serum. Three days after the transfection, the culture supernatants were collected. To each of the culture supernatants, fresh medium was added, and the culture was continued. Six days after the transfection, the culture supernatants were collected.

The amount of the recombinant proteins produced in the culture supernatants were evaluated by using IFN-αELISA kit (manufactured by BIOSOURCE INTERNATIONAL). The results are shown in Table 1.

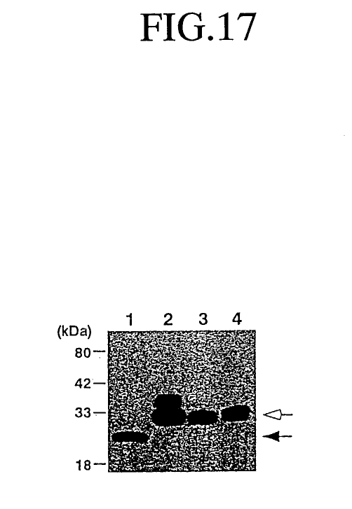

In addition, the recombinant proteins in the culture supernatants was subjected to Western blot analysis in the manner described above by using an anti-IFN-α monoclonal antibody (clone MMHA-2, PBL Biomedical). As shown in FIG. 17, it can be seen that each of IFN-α2b, IFN-α-CD81 fusion protein, IFN-α-bovine lactoferrin fusion protein and IFN-α-human lactoferrin fusion protein was detected in the culture supernatant of COS-1 cells transfected with each of pCIA, pCIA-CD81, PCIA-BLFN and pCIA-HLFN. By the above procedures, it was confirmed that IFN-α2b and HCV-binding type IFNs were transiently expressed in COS-1 cells. The interferon concentration in the resulting culture supernatants is shown in Table 1. When COS-1 cells were transfected with an empty vector pcDNA 3.1 to assay an anti-virus activity (described below), no anti-virus activity was found in the culture supernatant, so that it was confirmed that endogenous interferon is not induced.

TABLE 1
Interferon Concentration in Culture Supernatant of
Transfected COS-1 Cells

| Vector | Day 3 | Day 6 |
|---|---|---|
| pCIA | 936 ng/ml | 173 ng/ml |
| pCIA-BLFN | 654 ng/ml | 218 ng/ml |
| pCIA-CD81 | 976 ng/ml | 299 ng/ml |
| pCIA-HLFN | 258 ng/ml | 110 ng/ml |

(5) Anti-Virus Activity of Recombinant Protein

The anti-virus activities of IFN-α2b and HCV-binding type IFNs which had been prepared from transiently expressed in COS-1 cells were assayed by an assay system using the system for evaluating Sindbis virus-FL cells. Each of 200 IU/mL human interferon α2b (INTRON A, manufactured by Sherring-Plough/YAMANOUCHI PHARMACEUTICAL CO., LTD.) or the culture supernatants of COS-1 cells resulting from transient expression of IFN-α2b and HCV-binding type IFNs was two-fold serial dilution with a medium containing fetal bovine serum where 1000-fold dilution was regarded as a dilution fold 1. The resulting dilution was cultured together with the FL cells.

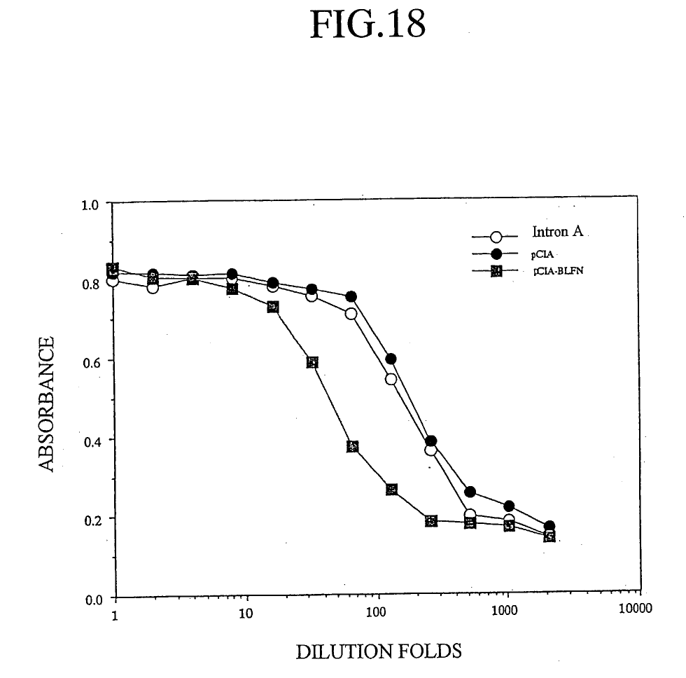

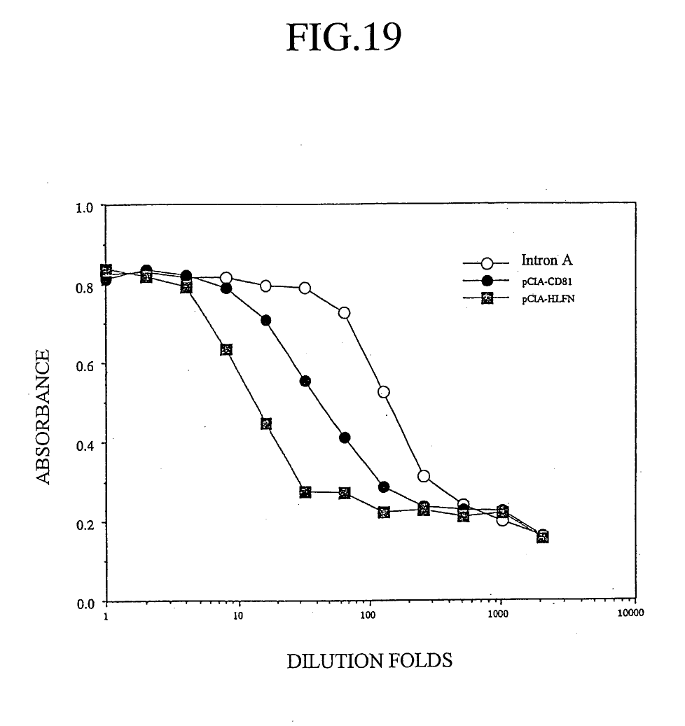

On the next day, Sindbis virus was added to the resulting culture, and the mixture was cultured for 2 days. Thereafter, the absorbance of the resulting culture at 595 nm was determined by Crystal Violet method. The determination was taken at n=2. In addition, at the same time, the FL cells alone were cultured as cell control, and the untreated FL cells added with Sindbis virus were cultured as virus control. The results are shown in FIG. 18 and FIG. 19.

It was confirmed from these results that IFN-α2b and HCV-binding type IFNs which had been transiently expressed in COS-1 cells have the anti-virus activity.

(6) Binding of HCV-Binding Type IFNs to HCV E2 Protein

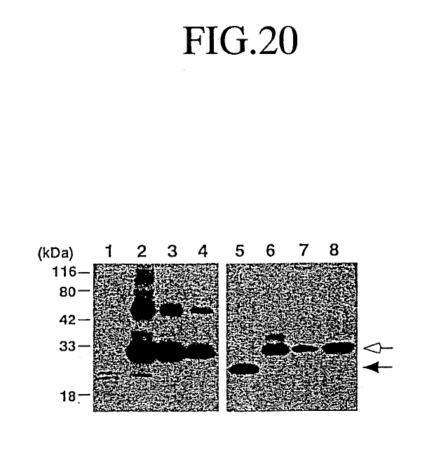

There was evaluated the binding of IFN-α2b or HCV-binding type IFNs which had been transiently expressed in COS-1 cells to the baculovirus-derived recombinant GST-fusion HCV E2 protein (hereinafter referred to as GST-E2; manufactured by Immuno Diagnostics) by coprecipitation using Glutathione beads. Five microliters of GST-E2 (1 μg/μl) was mixed with 400 μl of the culture supernatant of COS-1 cells containing transiently expressed IFN-α2b or HCV-binding type IFNs, and were incubated at 4° C. for 1 hour. Glutathione Sepharose 4B (Amersham Pharmacia) which had been incubated in buffer TBS-X [composition: 50 mM Tris-HCl (pH 7.5), 150 mM NaCl, 0.1% Triton X-100] containing 3% BSA for 30 minutes was added to a mixture of the culture supernatant of COS-1 cells and GST-E2, and the aliquots were incubated overnight at 4° C. After centrifugation, the supernatants (non-binding fraction) were collected, and the precipitated Glutathione beads were washed five times with the above-mentioned TBS-X. A 2× sample buffer was added to a bound fraction Glutathione beads, and the aliquots were treated at 100° C. for 5 minutes. Thereafter, the supernatants obtained by centrifugation were subjected to Western blot analysis using an anti-IFN-α antibody. Similarly, the supernatants of the non-binding fraction were subjected to Western blot analysis. The Western blot analysis was carried out in the same manner as in item (3) above. As shown in FIG. 20, HCV-binding type IFNs were coprecipitated with GST-E2, but IFN-α2b was not. It was suggested from these results that HCV-binding type IFNs bind to GST-E2 although IFN-α2b itself does not bind to GST-E2.

### EXAMPLE 6

Identification of Binding Sequence of HBV to Pre-S

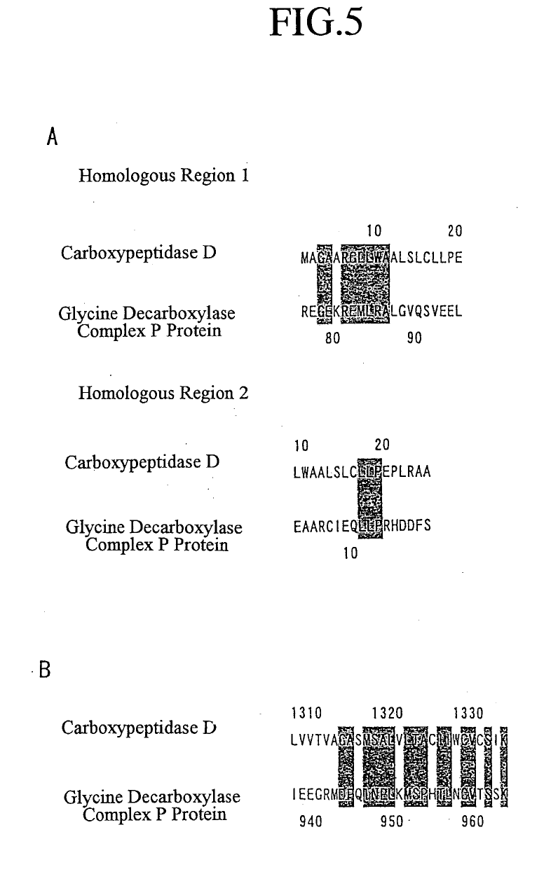

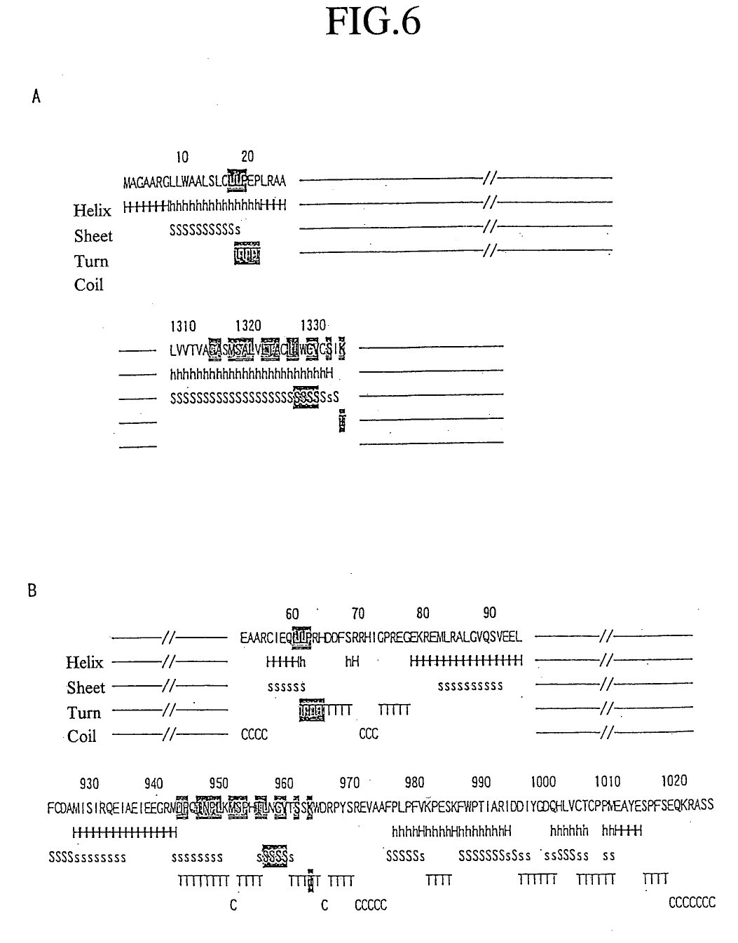

A binding region was analyzed, on the bases of an amino acid sequence of duck-derived carboxypeptidase D of SEQ ID NO: 14, and an amino acid sequence of duck-derived glycine decarboxylase complex P protein of SEQ ID NO: 15, which had been reportedly bound to duck HBV pre-S antigen. In the case of duck-derived carboxypeptidase D, 25 amino acids including N-terminal signal sequence and a region of 1307th amino acid residue to 1333rd amino acid residue (amino acid NOs: 1307 to 1333 in the amino acid sequence disclosed in accession No. AF039749) have been considered to be important for binding to pre-S. In addition, in the duck-derived glycine decarboxylase complex P protein, a sequence consisting of 52nd amino acid residue to 94th amino acid residue (amino acid NOs: 52 to 94 in the amino acid sequence disclosed in accession No. AF137264), and a sequence consisting of 923rd amino acid residue to 1024th amino acid residue, the C-terminal (amino acid NOs: 923 to 1024 disclosed in the above-mentioned accession No. AF137264) are considered to be necessary for binding. Therefore, the homologies between the amino acid sequences of respective regions were studied. The results are shown in FIG. 5. In the study of the homologies of N-terminal side sequence, a homologous region 1, which is thought to include a signal peptide of carboxypeptidase D was selected. However, since a signal sequence usually consists of 11 to 13 amino acids, the homologous region 1 is hardly likely to be a binding region for HBV receptor. Probably, the LLP sequence of a homologous region 2 is thought to be important. In addition, in the analysis on C-terminal side, the homologies were found in a region consisting of 1313th amino acid residue to 1333rd amino acid residue of carboxypeptidase D (amino acid NOs: 1313 to 1333 in the amino acid sequence disclosed in accession No. AF039749) and a region consisting of 943rd amino acid residue to 963rd amino acid residue of glycine decarboxylase complex P protein (amino acid NOs: 943 to 963 in the amino acid sequence disclosed in accession No. AF137264). Next, when a secondary structure of each homologous region was predicted, especially in the analysis of a similar region on C-terminal side, it was clarified that only in the region where identical amino acids are present in a large number takes the same tertiary structure. The results of prediction of the secondary structure are shown in FIG. 6.

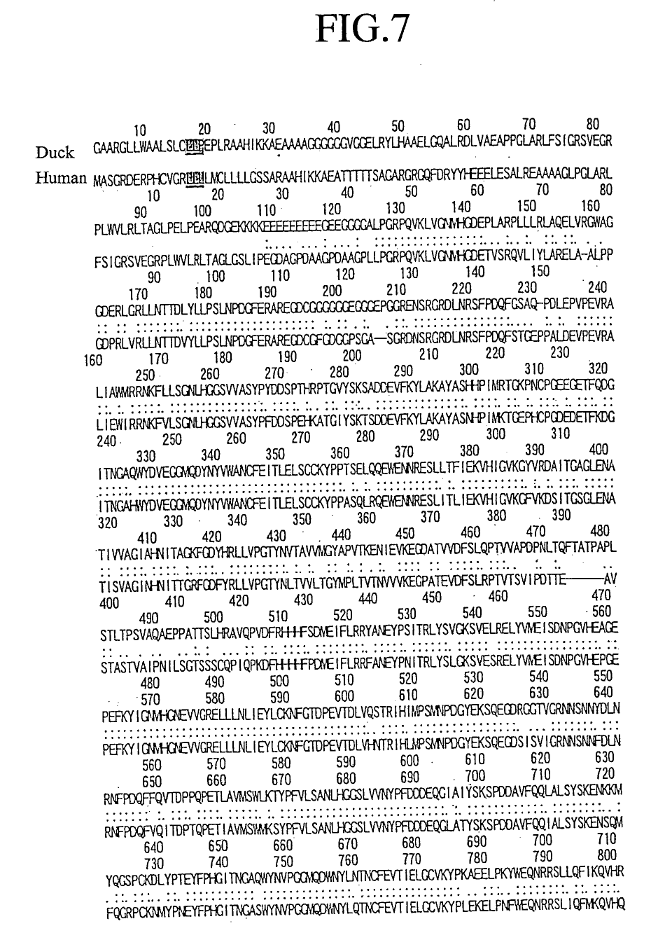

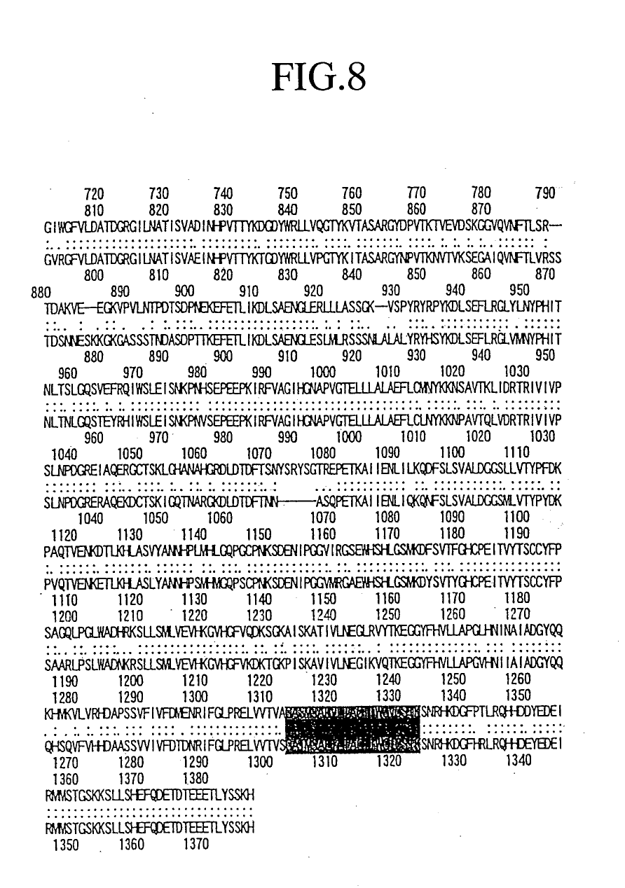

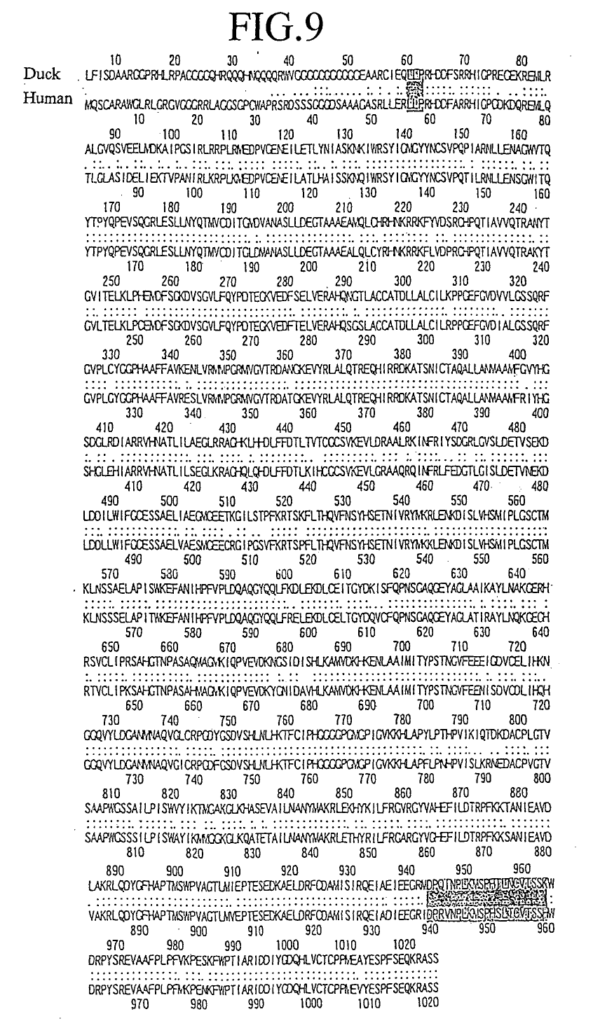

A binding region to duck HBV obtained in each duck-derived gene was compared with an amino acid sequence of human-derived carboxypeptidase D of SEQ ID NO: 16 and an amino acid sequence of human-derived glycine decarboxylase complex P protein of SEQ ID NO: 17. The evaluation results for homologies of each of the genes of duck and human are shown in FIG. 7, FIG. 8 and FIG. 9. Although the high homologies were not found in a region which is thought to be an HBV-binding region of each carboxypeptidase D, other regions were well conserved in duck and human genes. Binding deduced regions for HBV are shown in FIG. 7, FIG. 8 and FIG. 9. It is deduced from the results that a region acquiring the affinity for HBV is C-terminal side sequence.

Sequence Listing Free Text

SEQ ID NO: 23 shows a sequence of a synthesized peptide of human CD81- derived peptide TAPA148.

SEQ ID NO: 24 shows a sequence of a synthesized peptide of bovine lactoferrin-derived peptide bLF443.

SEQ ID NO: 25 shows a sequence of a synthesized peptide of human lactoferrin-derived peptide hLF445.

SEQ ID NO: 26 shows a sequence of a synthesized peptide of human LDL receptor-derived peptide hLDLR194.

SEQ ID NO: 27 shows a sequence of a synthesized peptide of human LDL receptor-derived peptide hLDLR220.

SEQ ID NO: 28 shows a sequence of a synthesized peptide of bovine lactoferrin-derived cyclic peptide cbLF445.

SEQ ID NO: 29 shows a sequence of a synthesized oligonucleotide for interferon-α2b-specific primer U-2.

SEQ ID NO: 30 shows a sequence of a synthesized oligonucleotide for interferon-α2b-specific primer L2.

SEQ ID NO: 31 shows a sequence of a synthesized oligonucleotide for H3TER primer.

SEQ ID NO: 32 shows a sequence of a synthesized nucleotide having a sequence corresponding to the deduced HCV E2 binding region in CD81.

SEQ ID NO: 33 shows a sequence of a synthesized nucleotide having a sequence corresponding to the deduced HCV E2 binding region in bovine lactoferrin.

SEQ ID NO: 34 shows a sequence of a synthesized nucleotide having a sequence corresponding to the deduced HCV E2 binding region in human lactoferrin.

### Industrial Applicability

According to the prophylactic or therapeutic agent for chronic hepatitis of the present invention, it is considered that the binding of hepatitis virus, especially hepatitis C virus or hepatitis B virus, to a target cell is inhibited. Therefore, there are exhibited excellent effects that chronic hepatitis can be prevented or treated. Therefore, in the interferon treatment method for chronic type-C and type-C hepatitis, there are even more expected that the viral clearance ratio at the termination of the interferon administration can be improved by the suppression of the infection on a target cell for hepatitis virus. The improvement in the clearance ratio of the hepatitis virus at the termination of the interferon according to the present invention is clinically very effective, from the viewpoints such that the improvement not only leads to an increase in complete responders but also that the development of hepatocellular carcinoma can be suppressed.

1. (amended) A prophylactic or therapeutic agent for chronic type-C hepatitis, comprising an oligopeptide having at least one kind of an amino acid sequence selected from the group consisting of SEQ ID NOs: 1, 2, 3, 18, 19, 26 and 27.

2. (amended) The prophylactic or therapeutic agent for chronic type-C hepatitis according to claim 1, comprising an oligopeptide having 20% or more homology with at least one kind of an amino acid sequence selected from the group consisting of SEQ ID NOs: 1, 2, 3, 18, 19, 26 and 27 at the homologous amino acid level, wherein said oligopeptide has the following characteristics:
(A) having a binding affinity to a viral antigen protein;
(B) inhibiting binding of virus to a receptor protein on a target cell for said virus; and
(C) having homologies with said receptor protein at the amino acid sequence level.

3. (amended) The prophylactic or therapeutic agent for chronic type-C hepatitis according to claim 1 or 2, wherein said oligopeptide is an oligopeptide having the following characteristics:
I) consisting of 31 to 33 amino acids which are flanked by cysteine residues;
II) forming loop structure: and
III) comprising basic amino acid residues at a middle portion and C-terminal side of the loop structure.

4. (amended) The prophylactic or therapeutic agent for chronic type-C hepatitis according to claim 1 or 2, wherein said oligopeptide is a cyclic oligopeptide comprising an oligopeptide having the following characteristics:
I) consisting of 31 to 33 amino acids which are flanked by cysteine residues;
II) forming loop structure: and
III) comprising basic amino acid residues at a middle portion and C-terminal side of the loop structure.

5. (amended) A prophylactic or therapeutic agent for chronic type-C hepatitis, comprising a nucleic acid having at least one kind of a nucleotide sequence selected from the group consisting of SEQ ID NOs: 4, 5 and 6.

6. (amended) A prophylactic or therapeutic agent for chronic type-C hepatitis, comprising a nucleic acid having at least one kind of a nucleotide sequence selected from the group consisting of SEQ ID NOs: 20 and 21.

7. (amended) A prophylactic or therapeutic agent for chronic type-B hepatitis, comprising a compound having the following characteristics:
(a′) having a binding affinity to S antigen protein of HBV; and
(b′) inhibiting binding of HBV to a receptor protein on a target cell for said HBV.

8. (amended) The prophylactic or therapeutic agent for chronic type-B hepatitis according to claim 7, comprising an oligopeptide having an amino acid sequence of SEQ ID NO: 10 or 11.

9. (amended) A prophylactic or therapeutic agent for chronic type-B hepatitis, comprising a nucleic acid having a nucleotide sequence of SEQ ID NO: 12 or 13.

10. (amended) A fusion interferon containing an amino acid sequence of an oligopeptide having the following characteristics:
(A) having a binding affinity to a viral antigen protein;
(B) inhibiting binding of virus to a receptor protein on a target cell for said virus; and
(C) having homologies with said receptor protein at the amino acid sequence level.

11. (amended) A fusion interferon containing at least one kind of an amino acid sequence selected from the group consisting of SEQ ID NOs: 1, 2, 3, 26 and 27.

12. (amended) A fusion interferon containing at least one kind of an amino acid sequence selected from the group consisting of SEQ ID NOs: 18 and 19.

13. (amended) A fusion interferon containing an amino acid sequence of SEQ ID NO: 10 or 11.

14. (amended) A method for treating chronic hepatitis in combination with the use of interferon, characterized by using an oligopeptide in combination with interferon, wherein said oligopeptide has the following characteristics:
(A) having a binding affinity to a viral antigen protein;
(B) inhibiting binding of virus to a receptor protein on a target cell for said virus; and
(C) having homologies with said receptor protein at the amino acid sequence level.

15. (deleted)

16. (deleted)

17. (deleted)

18. (deleted)
